(19) United States
(12) Patent Application Publication
(10) Pub. No.: US 2016/0317654 A1
(43) Pub. Date: Nov. 3, 2016

(54) COMBINATION THERAPY WITH RAR ALPHA AGONISTS FOR ENHANCING TH1 RESPONSE

(71) Applicant: King's College London, London (GB)

(72) Inventors: Randolph J. Noelle, Plainfield, NH (US); Graham M. Lord, London (GB); Chrysothemis C. Brown, London (GB)

(73) Assignee: Kings College London, London (GB)

(21) Appl. No.: 15/064,412

(22) Filed: Mar. 8, 2016

Related U.S. Application Data
(60) Provisional application No. 62/130,240, filed on Mar. 9, 2015.

(51) Int. Cl. A61K 39/39; A61K 39/395; A61K 35/17; A61K 31/196; A61K 31/203; A61K 31/428; A61K 31/69; A61K 39/00; A61K 45/06

(52) U.S. Cl. CPC A61K 39/39; A61K 39/0011; A61K 39/39558; A61K 35/17; A61K 45/06; A61K 31/203; A61K 31/428; A61K 31/69; A61K 31/196; A61K 2039/55511; A61K 2039/57; A61K 2035/124

(57) ABSTRACT

Encompassed are methods of potentiating anti-tumor immunity comprising administering an RARα agonist to a patient having a tumor in combination with at least one other treatment and methods of suppressing a Th17 response in a patient comprising administering an RARα agonist in combination with at least one other treatment.

This application claims priority to U.S. Provisional Application No. 62/130,240, which was filed on Mar. 9, 2015, and which is incorporated by reference in its entirety.

This application contains a sequence listing submitted in electronic format. The file name is “20160330_01166-0001-00US_SeqList_ST25.txt,” it was created on Mar. 30, 2016, and is 5,014 bytes in size.

### FIELD

Treatment of cancer and autoimmune diseases using immunotherapy

### BACKGROUND

Immunotherapeutic strategies for targeting malignant disease are an active area of translational clinical research, and have been for several decades. While some positive test data has been shown with prior approaches, additional clinically-effective therapeutic strategies should be explored. The art especially desires cancer treatments that will apply to a broader cross-section of patients than presently-available therapies. Likewise, more effective treatments for autoimmune diseases are also desired.

The immune-oncology (I-O) community is seeking approaches and therapeutics that will enhance the efficacy of PD-1/CTLA-4/vaccine targeted therapies. These therapeutics are known to drive productive CD4+ and CD8+ T-cell responses to tumor antigens, leading to clinical benefit in cancer patients. The novel discovery described herein is that RARα agonists drive Th1 CD4+ T-cell responses, and their use as monotherapy or in combination with other I-O agents is distinct from the use of RARα agonists as direct tumor cell differentiation agents.

Vitamin A and its derivatives (retinoids) are agonists at retinoic acid receptors, and have activity in cellular growth, differentiation and apoptosis. There are three retinoic acid receptors (RAR-α, β, and γ), and these receptors form heterodimers with members of the complementary retinoid X receptor family (RXR-α, β, and γ). All-trans retinoic acid (ATRA) is an agonist at RAR receptors only. Bexarotene and 13-cis retinoic acid (RA) bind only to RXR receptors. ATRA and bexarotene have been approved for the treatment of human cancers.

ATRA, an RARα, β, and γ receptor agonist, has been used systemically to treat a subset of acute myeloid leukemia, specifically acute promyelocytic leukemia (APL) patients having an RARα translocation. In APL, the RARα gene is aberrantly fused to a fusion partner, typically the APL gene, and the resulting protein binds to DNA and recruits transcriptional co-repressors which impair granulocyte differentiation, key to the pathogenesis of leukemia. Treatment with ATRA causes the release of co-repressors from the DNA, releases repression of differentiation, and allows the granulocytes to differentiate normally. This treatment, however, is only indicated when the RARα translocation has occurred and thus has a very limited scope. This narrow indication dearly demonstrates that the utility of ATRA in AML relates to direct effects upon the fusion protein, and not to other effects upon T helper cells, which would not be limited to patients with fusion proteins in their tumor cells. One of the major limitations to the wide scale use of ATRA is its many, severe, toxicities, which may be due to its agonistic effects on RARβ or RARγ. As such a selective RARα agonist will have reduced toxicities and have broader utility. The toxicities observed with ATRA include the potentially fatal differentiation syndrome, cardiac toxicity and cutaneous toxicity.

ATRA previously failed to demonstrate activity in a breast cancer study when administered in combination with paclitaxel. Clinical studies of ATRA in lung cancer in combination with cytotoxic chemotherapy are underway, but these aim to exploit direct effects of ATRA upon cell death, most likely via stimulation of RARβ (typically measured as a biomarker), hence the use in combination with cytotoxic chemotherapy, which is recognized to generally suppress T-cell responses.

Bexarotene, a synthetic RXR agonist, bexarotene, is approved for the systemic treatment of cutaneous T-cell lymphoma (CTCL). Bexarotene has been tested clinically for activity in other human tumors but failed to show convincing evidence of activity in lung cancer (phase 3 trial in combination with chemotherapy) or breast cancer. 13-cis RA, another RXR agonist, has been tested in treatment of pre-malignant oral leukoplakia, and was shown to induce direct lesion shrinkage, but a meta-analysis suggested evidence was insufficient to support routine usage. 13-cis RA also failed to show compelling activity as monotherapy in breast cancer.

It is well established that Th1 CD4+ T-cells are important to the development of productive anti-tumor immunity, with interferon-γ, a critical Th1 cytokine, also implicated. In association with tumor-specific CD8+ cytolytic T-cells, promotion of Th1 CD4+ T-cell differentiation and stabilization has been widely shown to enhance anti-tumor immunity. The role of RAR in Th1 cell biology has been hitherto unclear, and implications for the treatment of cancer have been unrecognized. Only with the present work has that pathway been elucidated. Additionally, in the prior art, ATRA has been administered in combination with cytotoxic chemotherapy, which generally suppresses T-cell responses. Only with this discovery, it becomes clear that coadministration of ATRA or other RARα agonists with immunosuppressive cytotoxic agents actually reduces the beneficial impact of the RARα agonist, which generally suppress T-cell responses (i.e., suppresses or entirely prevents the previously unknown immunomodulatory effects from occurring). The approach of monotherapy with an RARα agonist, or combination use with immunomodulatory therapeutics, has not been described previously.

Certain retinoids have been attempted for use in treatment of autoimmune diseases, but have been limited by side effects and potential concerns regarding teratogenicity. With this study, we are now appreciating that the immune effects of ATRA and other RAR agonist occur through RARα, not RARβ or RARγ. As such, methods of treatment with agonists specific for RARα can provide benefit and exclude certain side effects associated with RARβ or RARγ.

Here we show that RA-RARα is useful for maintenance of the Th1 cell lineage. Loss of RA signaling in Th1 cells resulted in the emergence of hybrid Th1-Th17 and Th17 effector cells. Global analysis of RARα binding and enhancer mapping revealed that RA-RARα directly regulated enhancer activity at Th1 cell lineage-defining genes while repressing genes that drive Th17 cell fate. In the absence of RA signaling, infectious and oral antigen induced inflammation resulted in impaired Th1 cell responses with deviation towards a Th17 cell phenotype. These findings identify RA-RARα as a regulatory node that acts to sustain the Th1 cell response while repressing Th17 cell fate. Thus RARα agonists can used to treat cancer by promoting the Th1 cell response and also can be used to treat autoimmune diseases by repressing Th17 cells.

### SUMMARY

CD4⁺ T-cells differentiate into phenotypically distinct T helper cells upon antigenic stimulation. Regulation of plasticity between these CD4⁺ T-cell lineages is useful for immune homeostasis and prevention of autoimmune disease. However, the factors that regulate lineage stability are largely unknown. Here we investigate a role for retinoic acid (RA) in the regulation of lineage stability using T helper 1 (Th1) cells, traditionally considered the most phenotypically stable Th subset. We found that RA, through its receptor RARα, sustains stable expression of Th1 lineage specifying genes as well as repressing genes that instruct Th17 cell fate. RA signaling is useful for limiting Th1 cell conversion into Th17 effectors and for preventing pathogenic Th17 responses in vivo. Our study identifies RA-RARα as a component of the regulatory network governing maintenance and plasticity of Th1 cell fate and defines an additional pathway for the development of Th17 cells.

In accordance with the description, a method of potentiating anti-tumor immunity comprises administering an RARα agonist to a patient having a tumor, as well as providing at least one other therapy to the patient to treat the tumor. Such at least one other therapy may be chosen from administering a checkpoint inhibitor to the patient having a tumor, administering a vaccine to the patient having a tumor, and treating the patient with T-cell based therapy.

In another embodiment, a method of suppressing a Th17 response in a patient comprises administering an RARα agonist, as well as at least one other therapy, to the patient.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A-1F3. RA Controls the Balance Between Th1 and Th17 Effector Cells. (A) Splenic CD4⁺ T-cells from dnRara and wild-type littermate control mice (WT) mice. Numbers indicate percentage CD62^{lo}CD44^{hi} cells (top left) or CD62L^{hi}CD44^{lo} T-cells (bottom right) gated on CD4⁺ cells. (B) Frequency and total number (C) of CD62L^{lo}CD44^{hi} in the CD4⁺ T-cell population in WT and dnRara mice (n=3-4 per group). (D) Intracellular IFN-γ and IL-17A expression in splenic CD4⁺CD44^{hi} T-cells after stimulation with phorbol 12-myristate 13-acetate (PMA) and ionoymycin. (E) Statistical data from cells as in (D). (F) Quantitative real time PCR analysis of Tbx21, Rorc and Gata3 in splenic CD4⁺CD62^{lo}CD44^{hi} cells (as in 1A), sorted by flow cytometry. Data are from two or three independent experiments with similar results. Mean±SEM, *p<0.05; **p<0.01; ***p<0.001; ****p<0.0001. See also FIG. 9.

FIGS. 2A-2E2. RA Signaling Required for Th1 Cell Differentiation and Repression of Th17 Cell Fate in Th1 Cell Precursors. Sorted naïve CD4+‘T’-cells from dnRara or WT mice were cultured under Th1 conditions for 6 days. (A) Intracellular expression of IFN-γ and IL-17A following stimulation with PMA and ionomycin. (B) T-bet and RORγt expression. Grey histograms indicate staining for Tbx21^{-/-} (left panel) or isotype control antibody (right panel). Numbers show MFI. Numbers in quadrants represent percent T-cells in each. (C) Amount of IL-17A, IL-21, IL-22 and IL-10 in supernatants following restimulation of cells as in (A) with α-CD3 and α-CD28 for 24 h as measured by multiplex bead array. Triplicate culture wells. (D) Quantitative real time PCR analysis of Th1 and Th17 cell signature cytokine and TF genes following stimulation with PMA and ionomycin. (E) Naive CD4⁺ T-cells from dnRara-Ifng^{eYFP} and Ifng^{eYFP} mice were cultured under Th1 conditions. IFN-γ (eYFP⁺) cells were sorted on day 7 following stimulation with PMA and ionomycin. Heatmaps displaying the fold changes of genes that were differentially expressed (fold change>1.5, p<0.05) for selected cytokines or cytokine receptors (upper panel) and TFs (lower panel). Samples from three independent experiments. Representative data of at least three (A, B) or two (C-D) independent experiments. Mean±SEM. See also FIG. 10.

FIGS. 3A1-3G. RA Required for Late Phase T-bet Expression. (A) Naive CD4⁺ T-cells from dnRara and WT mice were differentiated under Th1 conditions with combinations of IFN-γ or IFN-γ antibody. T-bet expression analysed at the indicated timepoints. Histograms gated on CD4+ T-cells. (B) Flow cytometric analysis of STAT4 phosphorylation in naïve CD4⁺ T-cells from dnRara and WT mice differentiated under Th1 conditions. Cells analysed directly from culture after 3 days (left panel) or on day 6 following treatment with (solid lines) or without (dashed lines) 25 ng/ml IL-12 for 30 min (right panel). Shaded histogram displays pSTAT4 staining in cells cultured under Th0 conditions. (C) Cell surface expression of IL-12Rβ2 on day 6 of culture. (D) Quantitative real-time PCR analysis of Il12rb1 and Il12rb2 on day 6. (E) Quantitative real-time PCR analysis of Stat4 in Th1 polarised cells at indicated time points. Expression relative to naïve CD4+ T-cells. (F) Western-blot analysis of total STAT4 protein on day 6 of Th1 culture. (G) Naive CD4⁺ T-cells from dnRara-Ifng^{eYFP} and control mice were activated under Th1 conditions. Frequency of IFN-γ⁺ (eYFP⁺) cells at indicated timepoints, gated on viable CD4⁺. Data representative of two to three independent experiments. Mean±SEM. See also FIG. 11.

FIGS. 4A1-4B3. Loss of RA Signaling in Fully Committed Th1 cells Leads to Th1 Plasticity and Divergence Towards the Th17 Lineage. (A) Naive CD4⁺ T-cells from dnRara^{lsl/lsl} mice were differentiated under Th1 conditions. Th1 cells were transduced with TAT-Cre on days 5 and 7 and repolarised under Th1 conditions for a further 5 days. Intracellular expression of T-bet and RORγt. (B) Naive CD4⁺ T-cells from Ifng^{eYFP} mice were differentiated under Th1 conditions. IFN-γ (eYFP⁺) cells were sorted on day 7 and restimulated under Th1 conditions for 5 days in the presence of Veh or RAi. Intracellular expression of T-bet and RORγt. Data representative of two independent experiments. See also FIG. 12.

FIGS. 5A-K. RA-RARα Regulates Enhancer Activity at Th1 Lineage Associated Loci and Represses Th17 Genes. Naive CD4⁺ T-cells from WT and dnRara mice were cultured for 6 days under Th1 conditions prior to chromatin precipitation and transcriptional profiling. (A) ChIP-seq binding tracks at Tbx21 locus for RARα in WT Th1 cells and p300 binding, H3K27ac, H3K4me1 and H3K4me3 modifications in WT and dnRara Th1 cells. (B) Validation of the RARα binding regions in WT Th1 cells by ChIP-qPCR. Untr6 region serves as a negative control. Binding events per 1000 cells displayed as ‘Enrichment’. (C) The effects of dnRara expression on p300 and H3k27ac abundance at the Tbx21 locus were validated by ChIP-qPCR. (D) Quantitative real-time PCR analysis of Batf, Irf4 and Ir8 mRNA in naive CD4⁺ T-cells from dnRara or WT-cells differentiated under Th1 cell conditions for 0, 24, 48, 72 h. Mean±SEM, replicate wells. (E) Log 2 values of fold changes in gene expression as measured by microarray analyses. Average fold change depicted. (F) ChIP-seq binding tracks at Irf8 locus for cells as in (A). (G) Validation of RARα ChIP-seq regions by ChIP-qPCR. (H-J) ChIP analysis of p300 and H3K27ac at selected loci. (K) ChIP analysis of H3K27me3 at the RORc locus. Arab locus serves as a negative control. Data from three independent experiments (E) or representative of two independent experiments (B-D, G-K); Mean±SD unless noted otherwise. Abbreviation: pro., promoter. See also FIG. 13.

FIGS. 6A-6D2. RA Signaling Required to Prevent the Generation of Th17 Cells During Infection with L. monocytogenes. (A) Frequency of LLOp:I-A^{b} CD4⁺ T-cells isolated from spleen of dnRara and WT mice 7 days after infection with an attenuated strain of L. monocytogenes (Lm-2W). Gated on CD4⁺ T-cells. (B) Absolute numbers of LLOp:I-A^{b} CD4+ T-cells as in (A). (C) Intracellular T-bet and RORγt expression gated on LLOp:I-A^{b} CD4⁺ T-cells. (D) Intracellular staining for IFN-γ and IL-17A following stimulation of splenocytes with LLOp for 6 h, 7 days after infection with Lm-2W. Gated on CD4⁺ T-cells. Right panel shows statistical data pooled from 3 independent experiments (3-6 mice per group). Representative data of at least three (A, B), or two independent experiments (C). Mean±SEM. See also FIG. 14.

FIGS. 7A-7F3. Loss of RA signalling Causes dysregulated Th1 and Th17 Response and Increased Pathogenicity in a Model of Gut Inflammation. (A) Schematic illustration of the adoptive transfer experiment. (B) Intracellular expression of IL-17A and IFN-γ among CD4⁺ cells from the spleen (Sp), mesenteric lymph nodes (MLN) and lymphocytes from the lamina propria (LPL) of mice as in (A) 7 days after transfer. (C) Statistical data for frequency of IFN-γ⁺, IL-17⁺ and IFN-γ⁺IL-17⁺ cells as in (B) in MLN and Sp. (D) Percentile change of original body weight in Rag1^{-/-} recipients treated as in (A) (n=5-7 per group). Mean±SD. (E) Frequency of diarrhoea-free mice among Rag1^{-/-} recipients as in (A) (OTII recipients n=3, OT-II (dnRara) recipients n=5). (F) Frequencies of IL-17, IFN-γ and Foxp3 in CD4⁺ cells isolated from Sp, MLN, LPL and IELs of mice as in (A), 9 days after transfer (n=5-6 per group). Data from one experiment (B-C), pooled from two independent experiments (D, F), or representative of two independent experiments (E). Mean±SEM.

FIG. 8 provides a graphical summary. Retinoic acid (RA) is produced at sites of inflammation. In the presence of Th1 instructing cytokines, RA suppress the differentiation of naive CD4+ T-cells into Th17 cells, in part through induction of IRF8 expression and repression of IL-6RA. RA further stabilises the Th1 phenotype by maintaining T-bet expression and repressing Runx1.

FIGS. 9A-9B2 (related to FIG. 1). Expression of Foxp3 in CD4⁺ T-cells deficient in RA signalinkate7Eg. (A) Intracellular expression of Foxp3 in CD4⁺ T-cells from spleen, thymus and mesenteric lymph nodes (MLN) of wild-type littermate control (WT) and dnRara mice. (B) Total number of CD4⁺Foxp3⁺ T-cells in spleen (upper panel) and thymus (lower panel) of WT and dnRara mice. Data are representative of two independent experiments. Mean±SEM.

FIGS. 10A1-10E2 (related to FIG. 2). Proliferation and differentiation of CD4⁺ T-cells in the absence of RA signalling. (A) Naïve CD4⁺ T-cells from WT and dnRara mice were labeled with CellTrace™ and cultured under Th1 conditions for 5 days. Flow cytometry showing dye dilution, gated on viable CD4⁺ T-cells. (B) Cell-surface expression of CD44 and CD25 on naïve CD4+ T-cells from WT or dnRara mice cultured under Th1 conditions for 5 days. (C) Naïve CD4+ T-cell from WT and dnRara mice were cultured under Th0 or Th2 conditions for 6 days. Cells were analysed by flow cytometry for expression of intracellular RORγt. Gated on CD4⁺ T-cells. (D) Sorted naïve CD4⁺ T-cells from WT and dnRara mice were cultured under Th17 conditions for 6 days. Intracellular IL-17A and IFN-γ expression after stimulation with PMA and ionomycin. (E) CD4⁺ T-cells from dnRara-Ifng^{eYFP} and Ifng^{eYFP} mice were cultured under Th1 conditions. Quantitative real-time PCR analysis of Cxcr3 and Il12rb2 from IFN-γ (eYFP⁺) cells sorted on day 7. Samples from three independent experiments. Representative data from two to three independent experiments (A-D). Mean±SEM.

FIGS. 11A1-B (related to FIG. 3). STAT3 and STAT4 activity in dnRara Th1 differentiated cells. (A) How cytometric analysis of STAT3 and STAT4 phosphorylation in naïve CD4+ T-cells from dnRara and T mice differentiated under Th1 conditions. Cells analysed after 6 days following treatment with 25 ng/ml IL-12, 20 ng/ml IL-6 and 10 ng/ml IL-23 for 30 minutes. Dashed lines represent untreated cells. (B) Bar graph depicts ratio of pSTAT3/pSTAT4 signaling as assessed by MFI.

FIGS. 12A-12B2 (related to FIG. 4). Cytokine analysis following temporal inhibition of RA signalling in Th1 cells. (A) Naive CD4⁺ T-cells from dnRara^{lsl/lsl} mice were cultured under Th1 conditions. Th1 cells were transduced with TAT-Cre on days 5 and 7 and repolarised under Th1 conditions for a further 5 days. Intracellular expression of IFN-γ and IL-17A following PMA and ionomycin stimulation. (B) Naive CD4+ T-cells from Ifng^{eYFP} mice were differentiated under Th1 conditions. IFN-γ (eYFP⁺) cells were sorted on day 7 and recovered cells underwent secondary repolarisation in Th1 conditions for 5 days in the presence of Veh or RAi. Intracellular expression of IFN-γ and IL-17A following PMA and ionomycin stimulation. Data representative of two independent experiments.

FIGS. 13A1-F (related to FIG. 5). RA-RARα regulates enhancers at Th1 genes and represses Th17 lineage specifying genes. Naive CD4⁺ T-cells from dnRara and WT mice were cultured under Th1 conditions as in FIG. 5. After 6 days, ChIP was performed with the specified antibodies, followed by real-time PCR analysis at selected sites (B-C) or sequencing (A). (A) ChIP-seq binding tracks at Stat4 and Ifng loci for RARα in WT Th1 polarised cells and p300 binding, H3K27ac, H3K4me1 and H3K4me3 modifications in WT and dnRara Th1 cells. (B) Validation of the RARα ChIP-seq regions in (A) by ChIP-qPCR assays. Untr6 region serves as a negative control. Data presented normalised to input. (C) Chip analysis of the abundance of p300 at the loci in (B) in WT and dnRara Th1 cells. Data presented normalised to input. (D) ChIP-seq analysis of STAT4 binding at the Tbx21 enhancer and comparison of p300 binding in WT and STAT4^{-/-} Th1 cells. ChIP-Seq data (Vahedi et al. 2012 and Wei et al., 2010) was mapped to the December 2011 (GRCm38/mm10) mouse genome assembly with the UCSC genome browser along with the ChIP-seq binding track for RARα at the Tbx21 locus. (E) Quantitative real time PCR analysis of selected genes identified as differentially expressed on genome wide transcriptional profiling analysis of cells as in (A). Mean±SEM. (F) Cell-surface expression of IL6-Rα by flow cytometry in naïve dnRara and WT CD4⁺ T-cells at indicated timepoints. Grey histogram indicates staining for isotype control. Data (B-F) representative of two to three independent experiments. Mean±SD unless otherwise stated, **p<0.01; ****p<0.0001.

FIG. 14A1-C (related to FIG. 6). Cytokine production by dnRARα T-cells following infection with L. monocytogenes. (A) Splenocytes from dnRara and WT mice infected with Lm-2W were restimulated with LLOp for 24 h. Concentration of IFN-γ, IL-17A and IL-4 in supernatants was measured by multiplex bead array (Biorad). Data normalised to total numbers of CD4⁺ T-cells. n=3-4 mice per group. (B) Intracellular staining for IFN-γ and IL-4 following stimulation of splenocytes with LLOp for 6 h, 7 days after infection with L. monocytogenes. Gated on CD3⁺CD4⁺ T-cells. (C) Cell surface expression of IL-6Rα by flow cytometry on LLOp:I-A^{b} CD4+ T-cells isolated from spleen of dnRara or WT mice 7 days after infection with L. monocytogenes. Data from 4 pooled mice. Numbers indicate MFI. Data representative of two to three independent experiments. Mean±SEM.

FIG. 15 (related to FIG. 7). Gut homing in dnRara-OTII CD4+ T-cells. Percentage of OTII or OTII(dnRara) CD4⁺ cells recovered from LPL, IEL, MLN and Spleen of RAG−/− recipients, 9 days after adoptive transfer (n=3-4 per group). Data representative of two independent experiments. Mean±SEM.

### DESCRIPTION OF THE SEQUENCES

Table 1 provides a listing of certain sequences referenced herein.

TABLE 1
Description of the Sequences

| | | SEQ |
|---|---|---|
| | | ID |
| Description | Sequences | NO |
| Stat4_ + 105k F | TCCTCCICCCTTTGTTGTTC | 1 |
| Stat4 + 105k R | GGGCCTTAATCAACCATTTC | 2 |
| Stat4 Promoter F | AGAGGGCATACACCGAGAAC | 3 |
| Stat4 Promoter R | TCTAGGGAGCCAGCATCAAC | 4 |
| Tbx21 Promoter F | TCGCTTTTGGTGAGGACTG | 5 |
| Tbx21 Promoter R | GGTGGCAGGTTGACTCTTTC | 6 |
| Tbx21 -12k F | GCGGAAGAGGGAACTAACAC | 7 |
| Tbx21 -12k R | GGACCCGGAACCTATGTATG | 8 |
| Irf8 Promoter F | CAGAAGCTAGGGCTGGTGTC | 9 |
| Irf8 Promoter R | CACAGAACAGATCCCAAATGTC | 10 |
| Irf8 -11k F | CCTTAACCCCGGAACTGTAG | 11 |
| Irf8 -11k R | TGCTGTGCTTGCCTCTACTC | 12 |
| Il6ra Promoter F | TCCGCTTGAGTTTTGCTTTC | 13 |
| Il6ra Promoter R | CACTGACCTGCCTTCTACTTTAAC | 14 |
| Il6ra + 32k F | CAAAGCTAAAACCAGGAAATGAC | 15 |
| Il6ra + 32k R | AAAAGGTTCCATGTGATGTTG | 16 |
| Rorc Promoter | AGGAATTTGGGTGTGGTGAG | 17 |
| (Rorgt isoform) F | | |
| Rorc Promoter | CTGTCTTGGGTGGTGTCTTG | 18 |
| (Rorgt isoform) R | | |
| Runx1 Promoter 1 F | TGGAAGAGGAAGAAGCTGTG | 19 |
| Runx1 Promoter 1 R | CAAGAGAAGCCACCCCAAAC | 20 |
| Runx1 Promoter 2 F | TGCTGGGCTTACACTTCTGAC | 21 |
| Runx1 Promoter 2 R | TGGACCTCATAAACAACCACAG | 22 |
| IFNg + 28k F | CTTTGAGCCACTGATGGGTAG | 23 |
| IFNg + 28k R | GCCTCTCCACGTCTCTTCTTC | 24 |

### DESCRIPTION OF THE EMBODIMENTS

### I. RARα Agonists

RARα agonists may include any agent that activates RAR or sustains retinoic acid so that its activity at RAR increases. This includes both substances that initiate a physiological response when combined with a receptor, as well as substances that prevent the catabolism (or breakdown) of retinoids (for example, retinoic acid), allowing the signal from retinoic acid itself to increase. As a nonlimiting list, RARα agonists include, but are not limited to ATRA, AM580, AM80 (tamibarotene), BMS753, BD4, AC-93253, and AR7. Additional RARα agonists include those provided in US 2012/0149737, which is incorporated herein by references for its teaching of the chemical structure of additional RARα agonists. For example, an RAR agonist may include: compound of the following formula, or a pharmaceutically acceptable salt thereof:

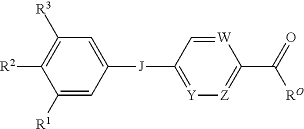

wherein: —R¹ is independently —X, —R^{X}, —O—R, —O—R^{A}, —O—R^{C}, —O-L-R^{C}, —O—R^{AR}, or —O-L-R^{AR}; —R² is independently —X, —R^{X}, —O—R^{X}, —O—R^{A}, —O—R^{C}, —O-L-R^{C}, —O—R^{AR}, or —O-L-R^{AR}; —R³ is independently —X, —R^{X}, —O—R^{X}, —O—R, —O—R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

In some embodiments, the RARα agonist is selective for RARα and does not produce significant agonistic effects on RARβ or RARγ. In some instances, about 100% or at least about 99%, 95%, 90%, 85%, 80%, 85° %, 80%, 70%, or 60% of the effect of the agonist impacts RARα as compared to combined impact on RARβ or RARγ.

In some embodiments, the RARα agonist is at least one substance that prevents the catabolism (or breakdown) of retinoids (for example retinoic acid), allowing the signal from retinoic acid itself to increase. Such agents may include retinioic acid metabolism blocking agents (RAMBAs), which are drugs that inhibit the catabolism of retinoids. RAMBAs temporarily raise the endogenous levels of all-trans-retinoic acid (all-trans-RA) in vivo. In doing so, they induce a local retinoid effect and avoid excessive systemic retinoid exposure, thereby avoiding some of the toxicity issues associated with retinoic acid agonists. RAMBAs will act as RARα agonists.

In some embodiments, RAMBAs include ketoconazol, liarozol, and/or tararozol.

### II. Methods of Treating Cancer

A method of potentiating anti-tumor immunity may be pursued by administering an RARα agonist to a patient having a tumor. In certain aspects, the method consolidates and/or maintains Th1 differentiated state in CD4+ and/or CD8+ T-cells. In some embodiments, a method of potentiating anti-tumor immunity comprises administering an RARα agonist together with an immune enhancer to a patient having a tumor.

In some embodiments, the patient does not have RARα translocated acute myeloid leukemia. In some embodiments, the patient does not have an RARα translocation. In some embodiments, the RARα agonist is not all-trans retinoic acid.

In some embodiments, the RARα agonist is administered without concomitant chemotherapy, such as without traditional small-molecule chemotherapeutic drugs, which would produce a cytotoxic effect that generally suppresses T-cell responses. For some patients, they have had no prior chemotherapy. For other patients, they have had no chemotherapy within at least about 2 weeks, 1, 2, or 3 months. For some patients, they will have no future chemotherapy within at least about 2 weeks, 1, 2, or 3 months, optionally so long as the RARα agonist shows treatment benefit.

Without being bound by theory, we have discovered that RARα agonists stabilize TH0 cells that are becoming TH1 cells, as well as provide for the maintenance of TH1 cells. Thus, this approach may be used for monotherapy or it may be used in combination with agents that trigger the TH0 to TH1 differentiation pathway.

A. Types of Cancer

In some embodiments, the cancer to be treated includes at least one of adrenocortical carcinoma; AIDS-related cancers (Kaposi sarcoma, lymphoma); anal cancer; appendix cancer; astrocytomas; atypical teratoid/rhabdoid tumor; basal cell carcinoma; bile duct cancer (e.g., extrahepatic bile duct cancer); bladder cancer; bone cancer; Ewing sarcoma family of tumors; osteosarcoma and malignant fibrous histiocytoma; brain stem glioma; brain cancer; central nervous system embryonal tumors; central nervous system germ cell tumors; craniopharyngioma; ependymoma; breast cancer; bronchial tumors; carcinoid tumor; cardiac (heart) tumors; lymphoma, primary; cervical cancer; chordoma; acute myelogenous leukemia (AML); chronic lymphocytic leukemia (CLL); chronic myelogenous leukemia (CAL); chronic myeloproliferative neoplasms; colon cancer; colorectal cancer; ductal carcinoma in situ (DCIS); embryonal tumors, endometrial cancer; esophageal cancer; esthesioneuroblastoma; extracranial germ cell tumor; extragonadal germ cell tumor; eye cancer (e.g., intraocular melanoma, retinoblastoma); fallopian tube cancer; gallbladder cancer; gastric (stomach) cancer; gastrointestinal carcinoid tumor; gastrointestinal stromal tumors (GIST); germ cell tumor (e.g., ovarian, testicular); gestational trophoblastic disease; glioma; hairy cell leukemia; head and neck cancer; hepatocellular (liver) cancer; hypopharyngeal cancer; islet-cell tumors, pancreatic cancer (e.g., pancreatic neuroendocrine tumors); kidney cancer (e.g., renal cell, Wilms tumor); Langerhans cell histiocytosis; laryngeal cancer; lip and oral cavity cancer; lung cancer (e.g., non-small cell, small cell); lymphoma (e.g., B-cell, Burkitt, cutaneous T-cell, Sézary syndrome, Hodgkin, non-Hodgkin); primary central nervous system (CNS); male breast cancer; mesothelioma; metastatic squamous neck cancer with occult primary; midline tract carcinoma involving nut gene; mouth cancer, multiple endocrine neoplasia syndromes; multiple myeloma/plasma cell neoplasm; mycosis fungoides; myelodysplastic syndromes; myelodysplastic/myeloproliferative neoplasms; nasal cavity and paranasal sinus cancer; nasopharyngeal cancer; neuroblastoma; oral cancer; oropharyngeal cancer, ovarian cancer (e.g., epithelial tumor, low malignant potential tumor); papillomatosis; paraganglioma; parathyroid cancer; penile cancer; pharyngeal cancer; pheochromocytoma; pituitary tumor; pleuropulmonary blastoma; pregnancy and breast cancer; primary peritoneal cancer; prostate cancer (e.g., castration-resistant prostate cancer); rectal cancer; rhabdomyosarcoma; salivary gland cancer; sarcoma (uterine); skin cancer (e.g., melanoma, Merkel cell carcinoma, nonmelanoma); small intestine cancer; soft tissue sarcoma; squamous cell carcinoma; testicular cancer; throat cancer; thymoma and thymic carcinoma; thyroid cancer; transitional cell cancer of the renal pelvis and ureter; cancer of unknown primary; urethral cancer; uterine cancer, vaginal cancer; vulvar cancer; or Waldenström macroglobulinemia.

In some embodiments, the cancer is acute myelogenous leukemia, bile duct cancer; bladder cancer; brain cancer; breast cancer; bronchial tumors; cervical cancer; chronic lymphocytic leukemia (CLL); chronic myelogenous leukemia (CML); colorectal cancer; endometrial cancer; esophageal cancer; fallopian tube cancer; gallbladder cancer; gastric (stomach) cancer; head and neck cancer; hepatocellular (liver) cancer; kidney (e.g., renal cell) cancer; lung cancer (non-small cell, small cell); lymphoma (e.g., B-cell); multiple myeloma/plasma cell neoplasm; ovarian cancer (e.g., epithelial tumor); pancreatic cancer; prostate cancer (including castration-resistant prostate cancer); skin cancer (e.g., melanoma, Merkel cell carcinoma); small intestine cancer; squamous cell carcinoma; testicular cancer; cancer of unknown primary; urethral cancer; uterine cancer.

B. Combination Therapy Approaches for Cancer

In certain aspects, the RARα agonist is administered in combination with at least one other therapy, such as an immuno-oncology agent, namely an immune enhancer.

In some embodiments, at least one other therapy promotes Th1 differentiation. At least one other therapy may be used to maintain Th1 immune response. At least one other therapy may be used to reintroduce Th1 immune response. In some aspects, the Th1 immune response is a Th1 immune response to an antigen expressed by the tumor.

In some embodiments, at least one other therapy is a Th1 differentiation therapeutic. A Th1 differentiation therapeutic may be chosen from at least one of, but is not limited to, IL-12, STAT-4, T-bet, STAT-1, IFN-γ, Runx3, IL-4 repressor, Gata-3 repressor, Notch agonist, and DLL.

In some aspects, at least one other therapy is a checkpoint inhibitor. For example the checkpoint inhibitor may be chosen from at least one of anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, CD40 agonist, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R checkpoint inhibitor. The checkpoint inhibitor may also cause STAT3 depletion. STAT3 depletion may be achieved through antisense technology or small molecule inhibitors, including cell surface receptor inhibitors, kinase inhibitors, and direct STAT3 inhibitors (including STAT3 SH2 domain inhibitors and STAT3 DNA-binding domain inhibitors). STAT3 inhibitors are described in Furtek et al, ACS Chem. Biol. 11:308-318 (2016), which is incorporated herein in its entirety for the disclosure of STAT3 inhibitors.

Optionally, a checkpoint inhibitor is an antibody. Such an antibody may be chosen from an anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, agonistic anti-CD40, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R antibody.

In some aspects, the checkpoint inhibitor helps to induce and/or maintain a therapeutic Th1 response.

In some embodiments, the at least one other therapy is a vaccine, containing one or more antigens expressed or likely to be expressed by a tumor. The vaccine may be based on a variety of delivery methodologies, including, but not limited to, peptides, DNA, RNA, viruses, virus-like particles, or cell-based vectors. Such a vaccine may be administered to stimulate the patient to produce T-cells or antibodies against the antigen, which would then mediate an immune response against the tumor. In such combination therapy the RARα agonist enhances the response to the antigens administered in the vaccine. For example, if the antigen was intended to induce a T-cell response, a co-administered RARα agonist would serve as a Th1-promoting “adjuvant” and would provide further therapeutic utility.

In some embodiments, the immuno-oncology agent is a bispecific antibody. In some embodiments, the immuno-oncology agent is a BITE (bispecific T-cell engaging antibody). In some embodiments, the bispecific antibody is anti-CD20 and anti-CD3; anti-CD3 and anti-CD19; anti-EpCAM and anti-CD3; or anti-CEA and anti-CD3.

In some embodiments, the combination therapy is a T-cell based therapy, such as an ex vivo cell based therapy. T-cell receptor technologies allow culturing or engineering of T cells with a T-cell receptor that can recognize a specific major histocompatibility complex (MHC) and peptide structure on a tumor. For example, a T-cell may be engineered to express an antibody or binding fragment thereof, where the antibody or fragment is specific for an antigen expressed by the tumor cell. This allows the T cells to target the patient's cancer cells. This culturing or engineering can be done ex vivo and the cells transplanted back into the patient to combine in the present methods. See Kim et al., Arch. Pharm. Res., DOI 10.1007/s12272-016-0719-7 (published online Feb. 19, 2016), which is incorporated herein in its entirety for the disclosure of T-cell receptor therapy.

C. Methods of Treating Autoimmune Diseases

In certain embodiments, a method of suppressing a Th17 response in a patient comprises administering an RARα agonist. Such a treatment may occur in a patient that has an autoimmune disease. In some embodiments, Th17 cells with an IFNg+ and/or IL17+ signature are suppressed.

Without being bound by theory, we have found that RARα agonist drive away from production of TH17 cells and towards TH1 cells.

D. Types of Autoimmune Diseases

In some aspects, the autoimmune disease is chosen from autoimmune diseases with an IFNg+IL17+ T-cell signature. In some embodiments, the autoimmune disease may be Juvenile Idiopathic Arthritis, Rheumatoid Arthritis, Crohn's disease, or Multiple Sclerosis.

In certain modes, the autoimmune disease is chosen from alopecia areata, autoimmune hemolytic anemia, autoimmune hepatitis, dermatomyositis, type 1 diabetes, juvenile idiopathic arthritis, glomerulonephritis, Graves' disease, Guillain-Barré syndrome, idiopathic thromnbocytopenic purpura, myasthenia gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, thyroiditis, uveitis, vitiligo, granulomatosis with polyangiitis (Wegener's).

In one embodiment, the autoimmune disease is not psoriasis and/or lupus.

E. Combination Therapy for Autoimmune Diseases

In certain embodiments, a combination therapy approach may be utilized by also administering one or more compounds that function to suppress T-cells, such as known treatments for autoimmune diseases.

Potential combination therapy agents include abatacept, adalimumnab, anakinra, azathioprine, certolizumab, certolizumab pegoltacrolimus, corticosteroids (such as prednisone), dimethyl fumarate, etanercept, fingolimod, glatiramer acetate, golimnumab, hydroxychloroquine, infliximab, leflunomide, mercaptopurine, methotrexate, mitoxantrone, natalizumab, rituximab, sulfasalazine, teriflunomide, tocilizumab, tofacitinib, vedolizumab.

Further aspects are provided through the following nonlimiting examples.

### EXAMPLES

### Example 1

### RA-RARα Regulates the Balance Between Th1 and Th17 Cells

To directly assess the role of RA in Th cell differentiation in vivo we used mice carrying a sequence encoding a dominant negative form of the RA receptor RARα (RARα 403) targeted to ROSA26 downstream of a loxP-flanked ‘stop’ (lsl) cassette.

C57Bl/6 dnRara mice have been described previously (Pino-Lagos et al., 2011). Mice were bred and maintained at Charles River Laboratory, UK in pathogen-free conditions. All animal experiments were conducted in accordance with the UK Animals (Scientific Procedures) Act 1986.

As shown previously (Pino-Lagos et al., 2011), interbreeding with mice expressing Cre recombinase from the Cd4 promoter generates Cd4^{cre}dnRara^{lsl/lsl} progeny (dnRara mice) in which RA signaling is abrogated within the T-cell compartment. In contrast to Rara^{-/-} mice, expression of this dnRARα disrupts the LA dependent activity of RARα while retaining the ligand independent effects, allowing the specific analysis of A dependent functions.

To investigate the role of RA in the generation of Th cell subsets under steady-state conditions, the expression of cytokines within CD4⁺ T-cells with an activated, CD44^{hi} phenotype was determined. Sort purified, naïve CD4⁺CD25-CD44^{lo}CD62L^{hi} T-cells were cultured with T-cell depleted splenocytes (APCs) and anti-CD3 under polarisation conditions for Th0, Th1, Th2 and Th17 cell-associated subsets.

Experimental conditions were as follows. Naïve CD4⁺CD25^{neg}CD44^{lo}CD62L^{hi} T-cells were isolated by cell sorting by FACSAria (BD) after enrichment with a CD4⁺ T-cell negative selection kit (Miltenyi Biotec). T-cell depleted splenocytes were prepared using a CD3⁺ microbead selection kit (Miltenyi Biotec) followed by irradiation at 3000 rad. Naïve CD4+ T-cells were cultured for 3 days with irradiated T-cell-depleted splenocytes at a ratio of 1:5 in the presence of 5 μg/ml of anti-CD3 (145-2C11) under Th0 cell conditions (IL-2 100 IU/ml, anti-IL-4 (11B11) and anti-IFN-γ (XMG 1.2), 10 μg/ml each); Th1 cell conditions (100 IU/ml of IL-2, 10 ng/ml of IL-12, and anti-IL-4); Th2 cell conditions (100 IU/ml of IL-2, 10 ng/ml of IL-4, anti-IL-12 (C17.8), and anti-IFN-γ (XMG 1.2); or Th17 cell conditions, 5 ng/ml TGFβ, 20 ng/ml IL-6, 10 ng/ml IL-1β, anti-IL-4, and anti-IFN-γ). Cells were expanded for an additional 3-4 days. Where indicated, 10 ng/ml IFN-γ or 10 μg/ml anti-IFN-γ was added. In secondary repolarisation assays, where specified, LE540 (1 μM) or DMSO (vehicle control) was added to the media. Cytokines were from R&D. Anti-CD3 was from BioXcell and other antibodies were from BD Biosciences. All cell cultures were performed in complete RPMI containing 10% fetal bovine serum (FBS), 55 M β-mercaptoethanol, HEPES, non-essential amino acids, glutamine, penicillin and streptomycin.

For analysis of cytokine production, cells were restimulated with 100 ng/ml phorbol 12-myristate 13-acetate (PMA) and 500 ng/ml ionomycin in the presence of monensin for 4-5 h at 37° C. in a tissue culture incubator. Cell surface staining was carried out in PBS with 2% FBS. For live cell analysis or cell sorting, dead cells were excluded by staining with SYTOX blue (Invitrogen). For intracellular staining, cells were first stained with LIVE/DEAD Fixable Violet or near IR Dead Cell Stain (Invitrogen), followed by staining for cell-surface markers and then resuspended in fixation/permeabilisation solution (Cytofix/Cytoperm kit or Transcription Factor Buffer kit; BD Bioscences). Intracellular staining carried out in accordance with the manufacturer's instructions. Intracellular phosphorylated STAT proteins were stained with Phosflow Lyse/Fix Buffer, and Phosflow Perm Buffer III (BD Biosciences) according to the manufacturer's protocol. Data were collected with a LSR Fortessa (BD) and results were analyzed with FlowJo software (Tree Star). All the antibodies for staining cell surface markers, cytokines or transcription factors were purchased from either BD Biosciences or eBiosciences.

Cytokine levels in supernatants were measured using a multiplex bead-based assay (Bio-Rad Laboratories) in a Luminex FlexMap3D System (Luminex Corporation).

Expression analysis was performed as follows. Total RNA was extracted from cells with RNeasy Mini kit (Qiagen) and cDN A was synthesized with Qscript RT kit (Quanta). Quantitative gene expression analysis was performed using Taqman primer probe sets (Applied Biosystems), listed in Table 2. Expression of target genes was normalized to β-actin.

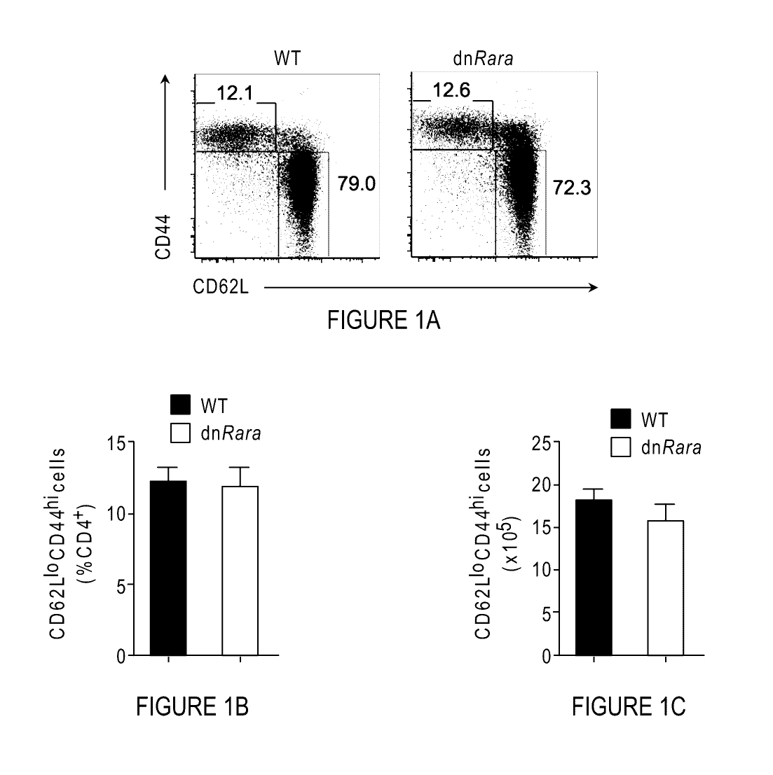

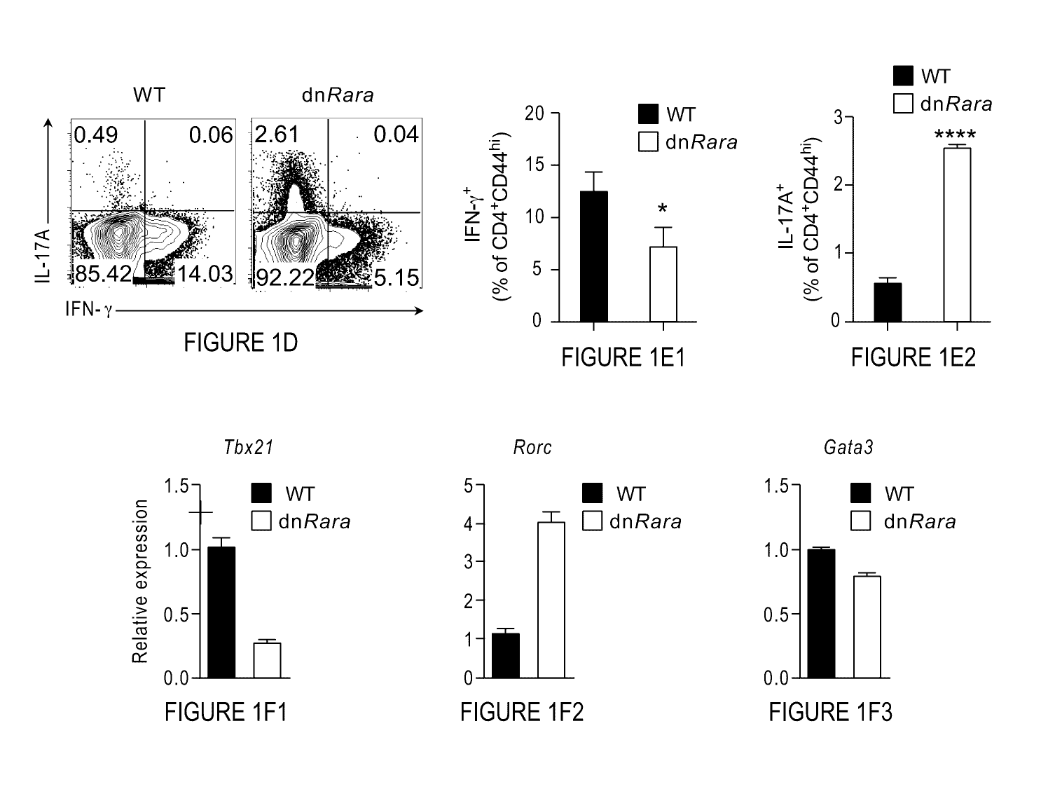

TABLE 2
Taqman assays used for RT-PCR gene expression
analyses (related to FIGS. 1-3 and 5).

| Mouse ACTB | 4352341E |
|---|---|
| Il6ra | Mm00439653_m1 |
| Il22 | Mm00444241_m1 |
| Runx1 | Mm01213404_m1 |
| Batf | Mm00479410_m1 |
| Cxcr3 | Mm99999054_s1 |
| Il23r | Mm00519943_m1 |
| Il1r1 | Mm00434237_m1 |
| Il21 | Mm00517640_m1 |
| Il10 | Mm00439616_m1 |
| Irf8 | Mm00492567_m1 |
| Irf4 | Mm00516431_m1 |
| Stat4 | Mm00448890_m1 |
| Il12rb2 | Mm00434200_m1 |
| Ifng | Mm00801778_m1 |
| Il12rb1 | Mm00434189_m1 |
| Rorc | Mm01261022_m1 |
| Gata3 | Mm00484683_m1 |
| Tbx21 | Mm00450960_m1 |

Sorted naïve CD4⁺ T-cells from dnRara or WT mice were polarised under Th1 conditions. On day 6 of culture cells were harvested and total RNA was extracted for microarray study or ChIP. RNA isolation, microarray and data processing performed by Miltenyi Biotec. For gene-expression analysis for the dnRara Th1 dataset Agilent microarray chips were used. Total RNA was extracted from cells lysed in Trizol LS reagent (Life Technologies). RNA quality was assessed with an Agilent 2100 Bioanalyzer (Agilent Technologies) and quantified with the Nanodrop ND-1000 UV-spectrophotometer (NanoDrop Technologies).

Transcriptome analysis was performed using Agilent Whole Mouse Genome Oligo Microarrays 8X60K in accordance with manufacturer's protocol. Data analysis was performed using R/bioconductor and software packages therein (www.R-project.org; wwv.bioconductor.org) or MS-Office Excel (Microsoft Inc.). Background corrected intensity values were normalized between arrays using quantile normalization. Quality controls include comparison of intensity profiles and a global correlation analysis. Differentially expressed genes were identified by statistical group comparisons on normalized (background corrected and quantile normalized) log 2 transformed fluorescence intensities using Student's t-test (two-tailed, equal variance). Reporters showing a p-value≦0.05 and a median fold-change in expression≧1.5 or ≦—1.5 were considered as reliable candidates for altered gene expression. In addition, at least two of the replicate samples in the group with higher expression were required to have detection p-values≦0.01.

Statistical significance was calculated by unpaired two-tailed Student's t test with Graphpad Prism software. p values<0.05 were considered significant. p values are denoted in figures by: *, p<0.05; **, p<0.01; ***, p<0.001; ****, p<0.0001.

Examination of the peripheral CD4⁺ T-cell compartment revealed equivalent frequencies and absolute numbers of CD44^{hi}CD62^{lo}CD4⁺ memory cells in 8-week old dnRara mice and in Cre⁻, wild-type, littermate controls (WT) (FIG. 1A-C). dnRara effector cells displayed reduced production of IFN-γ compared to their WT counterparts with a >5-fold increase in the frequency of IL-17⁺ cells (1D-1E2). Examination of transcripts for the signature lineage-determining TFs showed reduced mRNA expression of Tbx21 and significantly higher expression of Rorc in dnRara effector CD4+ T-cells (FIGS. 1F1-1F3). Loss of RA signaling had no impact on Th2 effectors with equivalent levels of Gata3 expression between dnRara and WT mice (FIGS. 1F1-1F3) and similar frequencies of IL-4 producing CD4⁺ T-cells (data not shown).

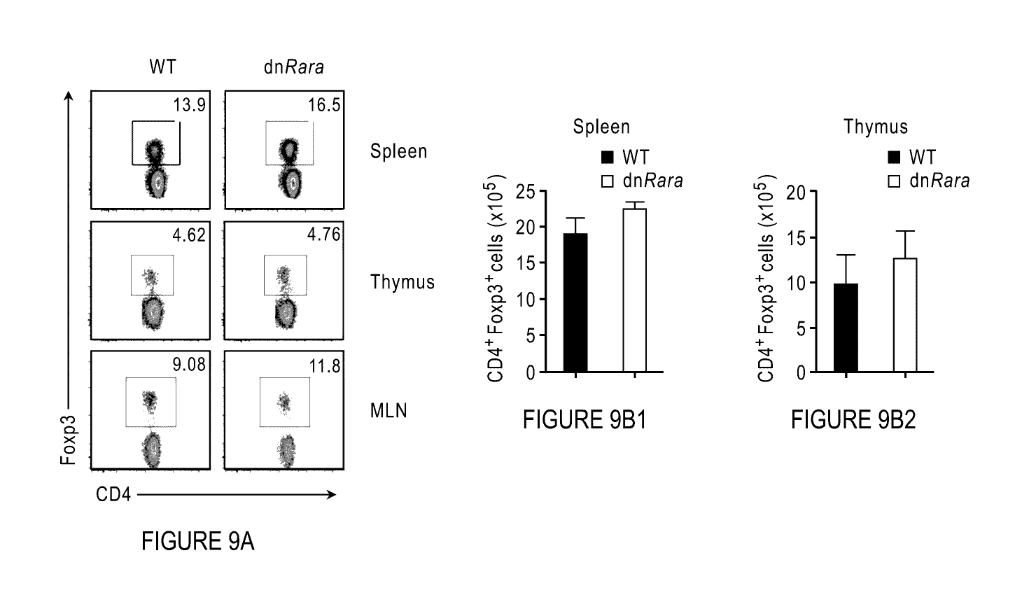

The frequency and numbers of Foxp3⁺ T-cells in the periphery and thymus of dnRara mice were similar to control mice (FIG. 9A-9B2), indicating that the increase in Th17 cells was not a consequence of reciprocal regulation by RA of Foxp3⁺CD4⁺ T-cells and Th17 cells (Mucida et al., 2007). Therefore, it is likely that under steady-state conditions RA is involved in differentiation of Th1 cells, while also limiting the differentiation of Th17 cells.

### Example 2

### RA Promotes Th1 Cell Differentiation and Inhibits Development of Th17 Cells from Th1 Cell Precursors

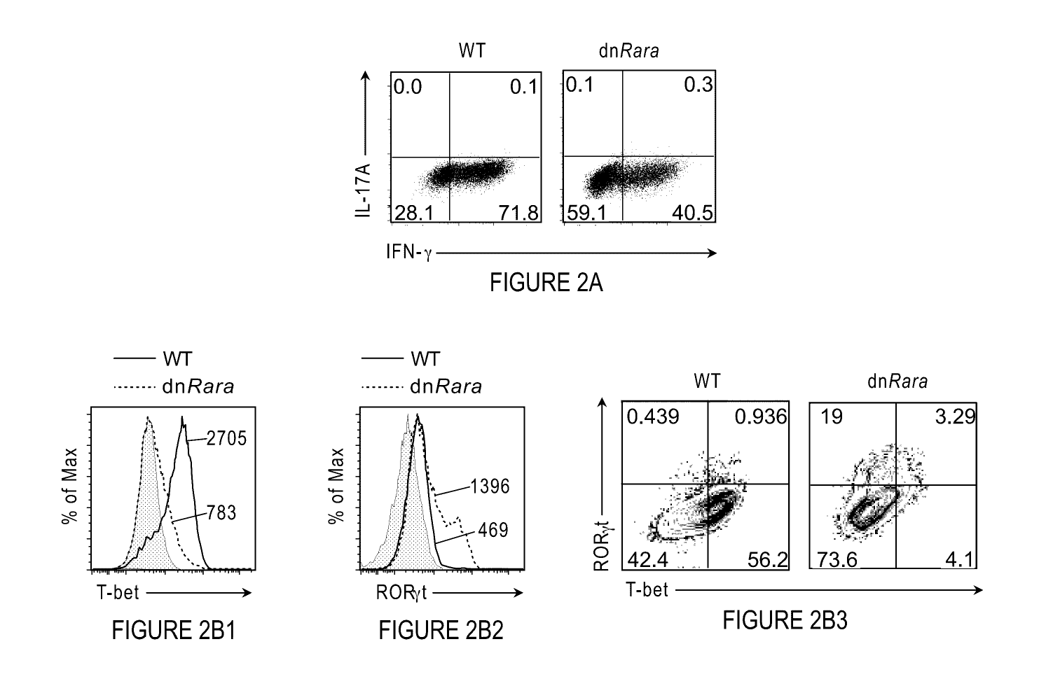

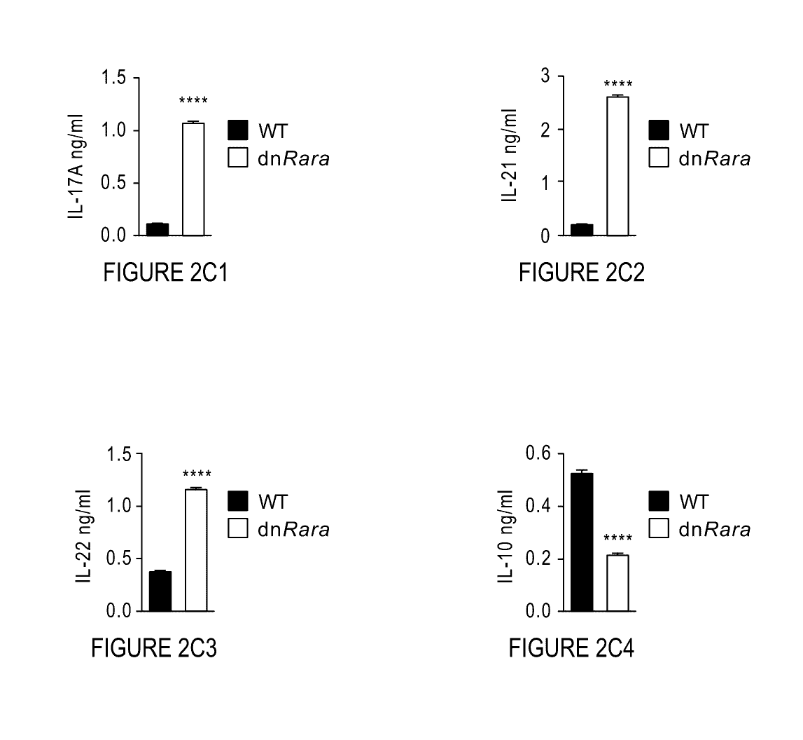

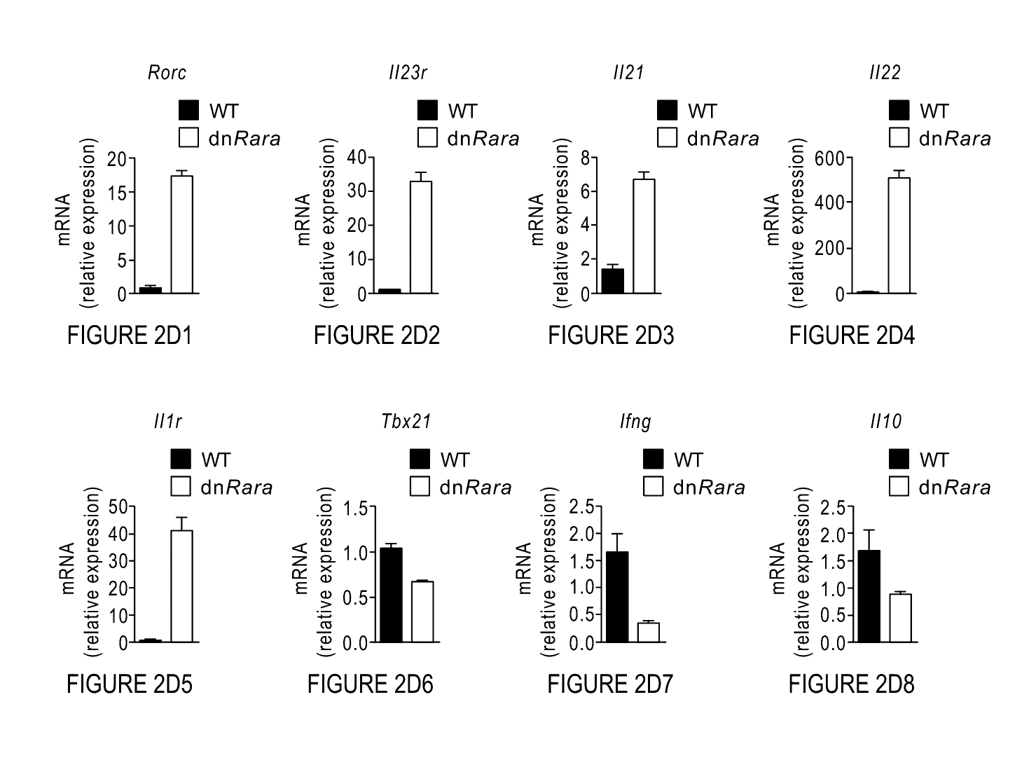

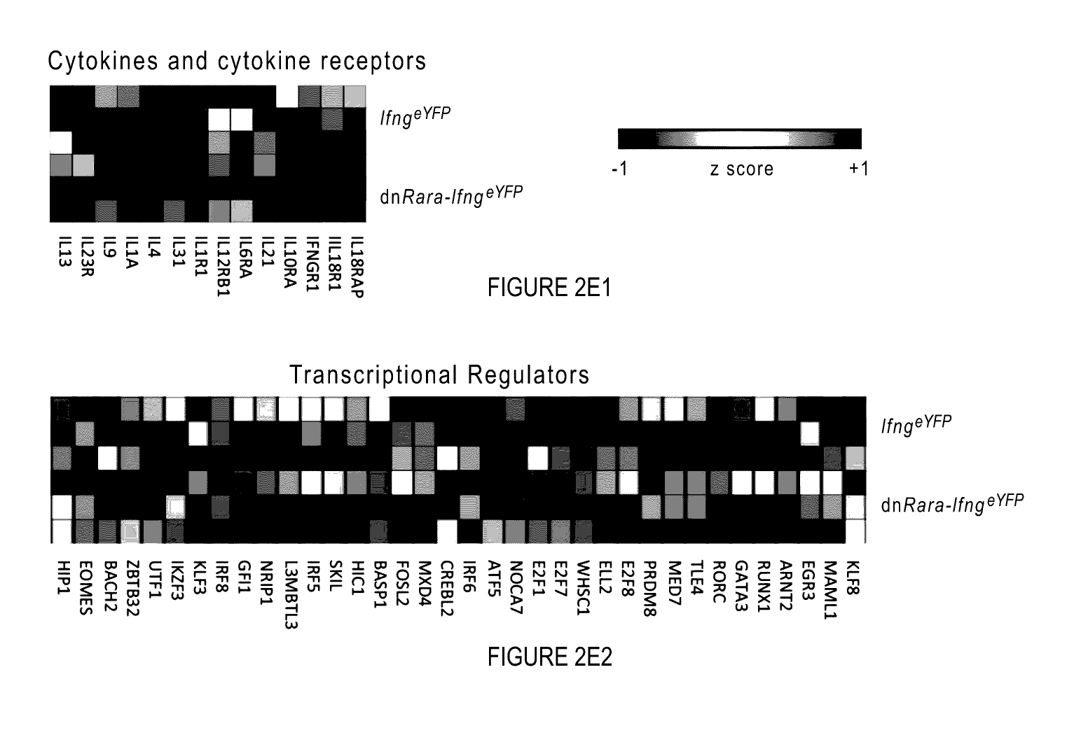

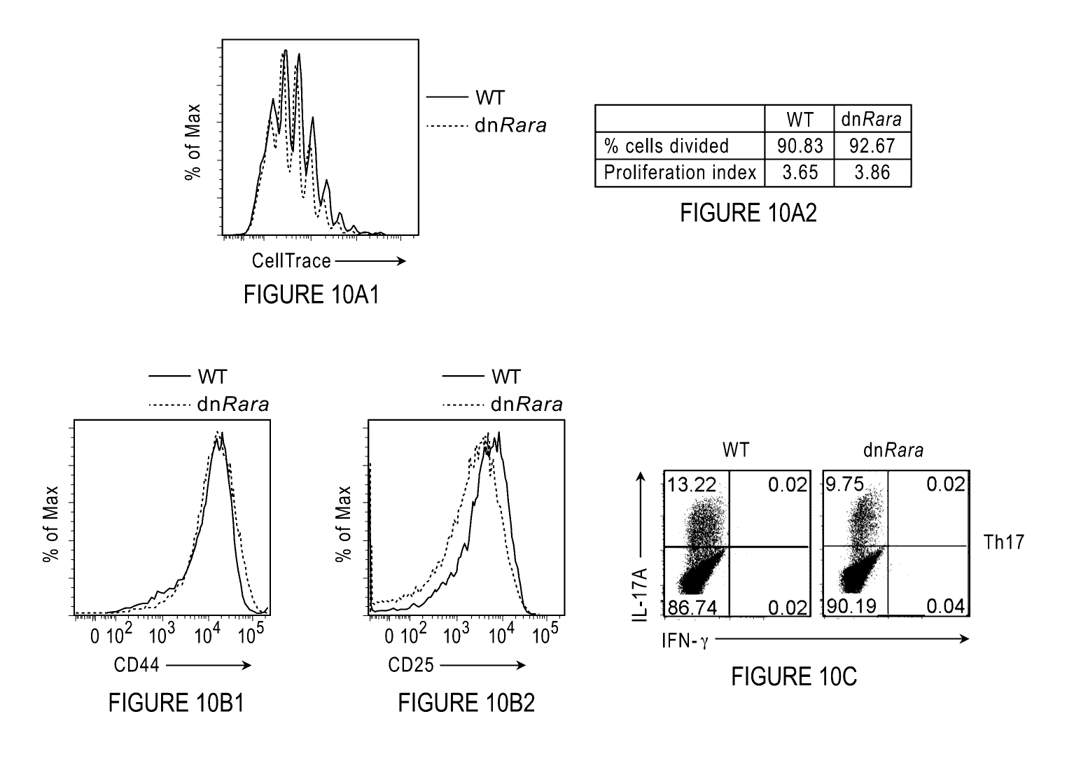

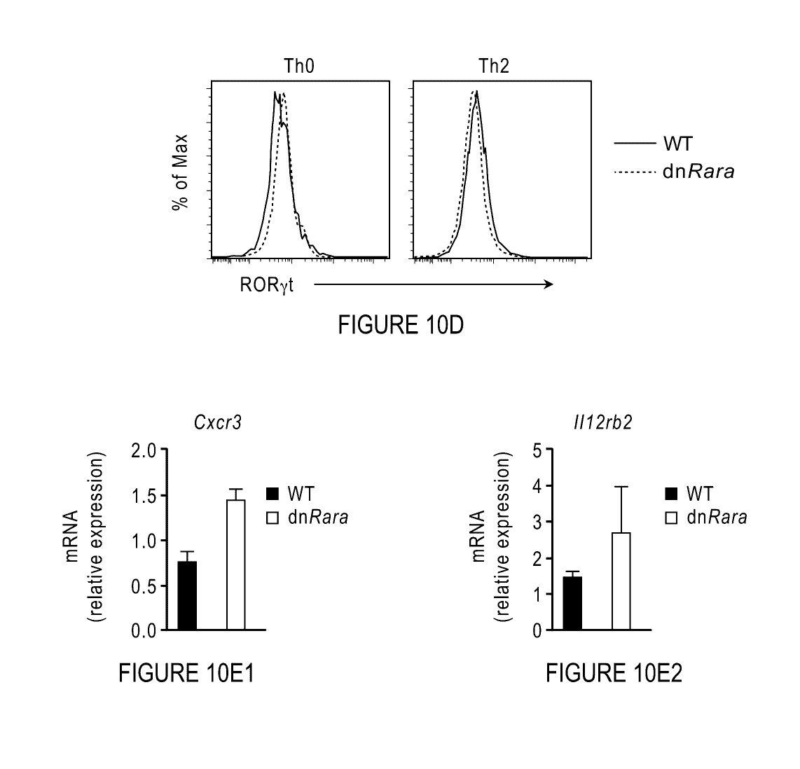

We considered two alternative explanations why dnRara mice exhibit reduced memory effector Th1 cells, in parallel with enhanced Th17 cells. The first possibility was that RA is required for the development of Th1 cells while independently suppressing the primary differentiation of Th17 cells. The alternative possibility was that RA is involved in restraining conversion of Th1 cells to Th17 cells. In order to resolve these two possibilities, naïve CD4⁺ T-cells were differentiated in the presence of Th1 or Th17 polarising cytokines. dnRara expressing CD4⁺ T-cells differentiated under Th1 cell conditions showed a markedly reduced capacity for IFN-γ production (FIG. 2A). Diminished cytokine production was not a consequence of impaired proliferative responses as naïve CD4⁺ T-cells differentiated under Th1 cell conditions showed robust proliferation, equivalent to WT-cells (FIG. 10A1-10A2). In addition, up-regulation of the activation markers CD25 and CD44 indicated that dnRara T-cells were not impaired in their ability to differentiate into effector cells (FIG. 10B1-10B2). Analysis of TF expression showed that ablating RA signaling resulted in a dramatic reduction in the expression of T-bet in CD4⁺ T-cells differentiated under Th1 cell conditions (FIG. 2B1-2B3). Strikingly, a substantial proportion of dnRara Th1 cells expressed RORγt and co-expression of T-bet and RORγt was observed at the single cell level. Although we did not observe intracellular IL-17A in cells following brief stimulation with phorbol myristate (PMA) and ionomycin, analysis of supernatants from Th1 polarised cells, reactivated on day 6 of culture on anti-CD3 and anti-CD28 coated plates for 24 h in non-polarising media, showed increased expression of IL-17A alongside other Th17 cell-associated cytokines (IL-21 and IL-22) (FIG. 2C1-2C4). Furthermore, mRNA analysis of dnRara Th1 polarised cells revealed dramatic increases in expression of certain signature Th17 cell genes (FIG. 2D1-2D8). Notably, these Th1 cells displayed the hallmarks of pathogenic Th17 cells with high amounts of Il23r expression but reduced amounts of IL10 mRNA and protein (FIG. 2C1-2C4 and FIG. 2D1-2D8) (Basu et al., 2013).

In order to assess whether enhanced Th17 responses were a general feature of CD4⁺ T-cells in which RA signaling is disrupted, naïve CD4⁺ T-cells from dnRara mice were differentiated under Th17 polarising conditions. In contrast to our observations above, we did not observe an increase in the frequency of IL-17⁺ cells in dnRara mice during primary differentiation into Th17 cells (FIG. 10C), suggesting that RA restrains Th17 cell differentiation only in the context of a Th1 polarising cytokine milieu. In support of this, RORγt expression was not observed in dnRara expressing naïve CD4⁺ T-cells differentiated under Th0 or Th2 conditions (FIG. 10D).

The simultaneous expression of RORγt and T-bet in dnRara Th1 cells suggested that RA-RARα might act to constrain the deviation of Th1 committed cells towards the Th17 cell lineage. To determine whether the RORγt⁺ cells represented a distinct T-cell population that arose directly from naïve CD4⁺ T-cells or from previously committed Th1 cells, Ifng^{eYFP} (Great) reporter mice were interbred with the dnRara mice to allow the tracking of IFN-γ⁺ cells.

Naïve CD4⁺ T-cells from dnRara-Ifng^{eYFP} or littermate control mice were activated under Th1 polarising conditions. Ifng^{eYFP} (GREAT) mice were purchased from the Jackson Laboratory. On day 7 of culture, following restimulation with PMA and ionomycin, eYFP⁺ cells were sorted and total RNA was extracted for transcriptional profiling using Affymetrix Mouse Gene 2.0 ST arrays. Pre-processing and statistical analysis of gene expression data were done using Partek Genomics Suite 6.6. CEL files were imported and expression intensities were summarised, normalised and transformed using Robust Multiarray Average algorithm. Two additional samples from eYFP⁺ dnRara or wild-type cells sorted without prior restimulation were included in the normalisation. These samples were not included in the analysis of differentially expressed genes. Differentially expressed genes were detected using fold-change and t-test analysis. P values<0.05 and fold change in expression ≧1.5 or ≦−1.5 were considered significant.

Certain signature Th17 cell genes, including Th17 cell cytokines and receptors for cytokines that promote Th17 cell differentiation (Il17f Il21, Il1r1, Il6ra, and Il23r), were highly expressed in dnRara IFN-γ expressing cells relative to WT mice, confirming a hybrid Th1-Th17 cell phenotype (FIG. 2E1-2E2). Of note, these Th1-Th17 cells retained high expression of Il12rb2 and Cxcr3 mRNA, equivalent to WT Th1 cells, while also expressing Il23r (FIG. 10E1-10E2). Genes associated with the Th2 cell subset such as Gata3 and Il4 were also dysregulated in dnRara Th1 cells consistent with a role for T-bet in repression of GATA3 (Zhu et al., 2012). These findings show that, in the absence of RA signaling, committed Th1 cell precursors can give rise to cells with a Th17 cell expression signature providing a new perspective on the origins of Th1-Th17 cells. Collectively these data demonstrate that RA is not only required for Th1 cell differentiation, but is also involved in suppressing Th17 cell development in Th1 polarised cells.

### Example 3

### RA-RARα is Required for Late Phase, STAT4 Dependent T-Bet Expression in Th1 Cells

Early expression of T-bet following TCR activation is dependent on IFN-γ, whereas late expression of T-bet (post-termination of TCR signaling) has been shown to be dependent on IL-12 (Schulz et al., 2009). To distinguish a requirement for RA signaling in Th1 cell commitment from maintenance of Th1 cell fate, we examined the kinetics of T-bet expression in naïve CD4⁺ T-cells cultured under Th1 polarising conditions.

Western blot analysis of differentiated Th1 cells was as follows. Differentiated Th1 cells were lysed in RIPA buffer supplemented with protease inhibitors. Lysates were electrophoresed on 10% gels (Biorad), transferred to nitrocellulose and blotted with anti-STAT4 or anti-actin followed by anti-rabbit-horseradish peroxidase conjugated antibody. All antibodies were from Cell Signaling Technology.

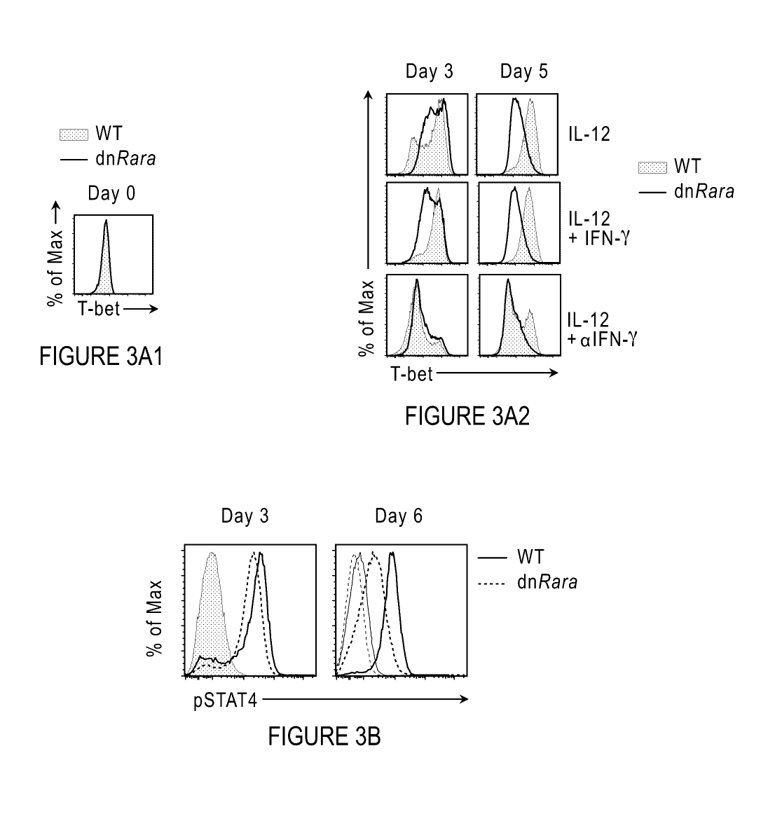

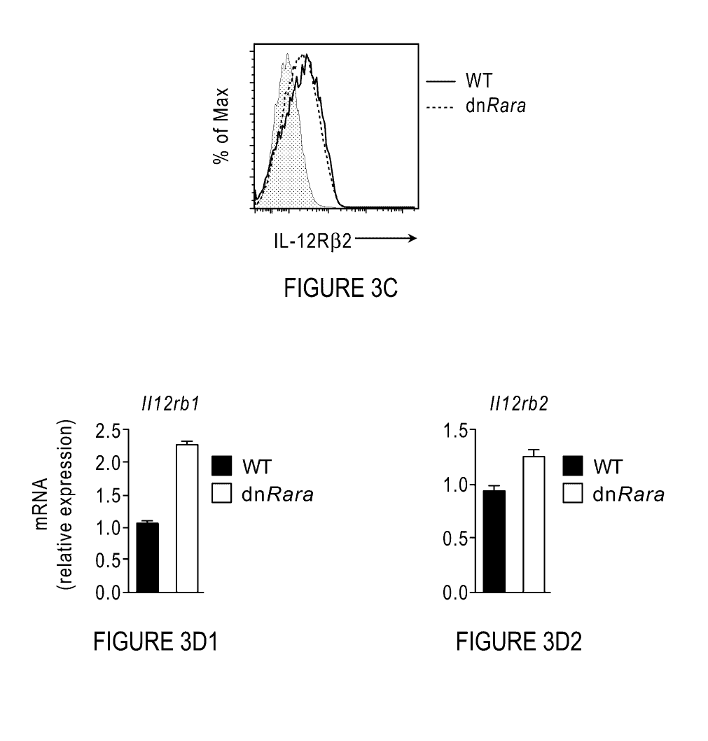

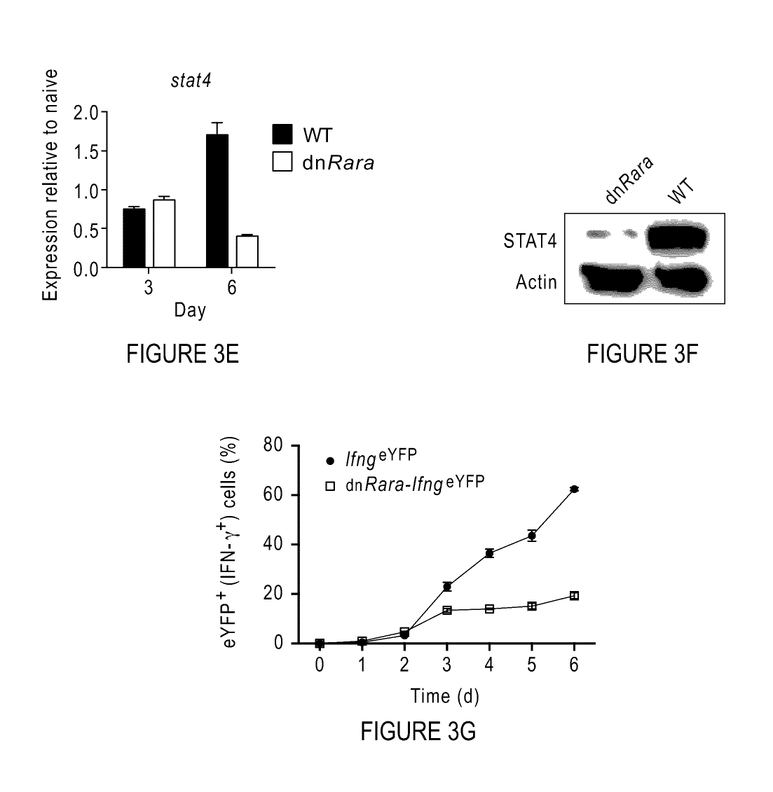

Induction of T-bet was observed with comparable amounts of T-bet expression between WT and dnRara T-cells at day 3 of culture, indicating that RA-RARα signaling is not required for early Th1 lineage commitment (FIG. 3A1-3A2). However, T-bet expression was not sustained in dnRara Th1 cells, with substantially diminished expression of T-bet by day 5 of culture. Given that IFN-γ promotes T-bet expression, the expression of T-bet was examined in the presence of recombinant IFN-γ, in order to avoid potential indirect effects caused by reduced IFN-γ production in dnRara Th1 cells. Exogenous IFN-γ enhanced early T-bet expression in both dnRara and WI Th1 cells but did not rescue the late (>72 h) impairment in T-bet expression (FIG. 3A1-3A2). IFN-γ signaling, as measured by STAT1 phosphorylation, was not impaired at either timepoint (data not shown).

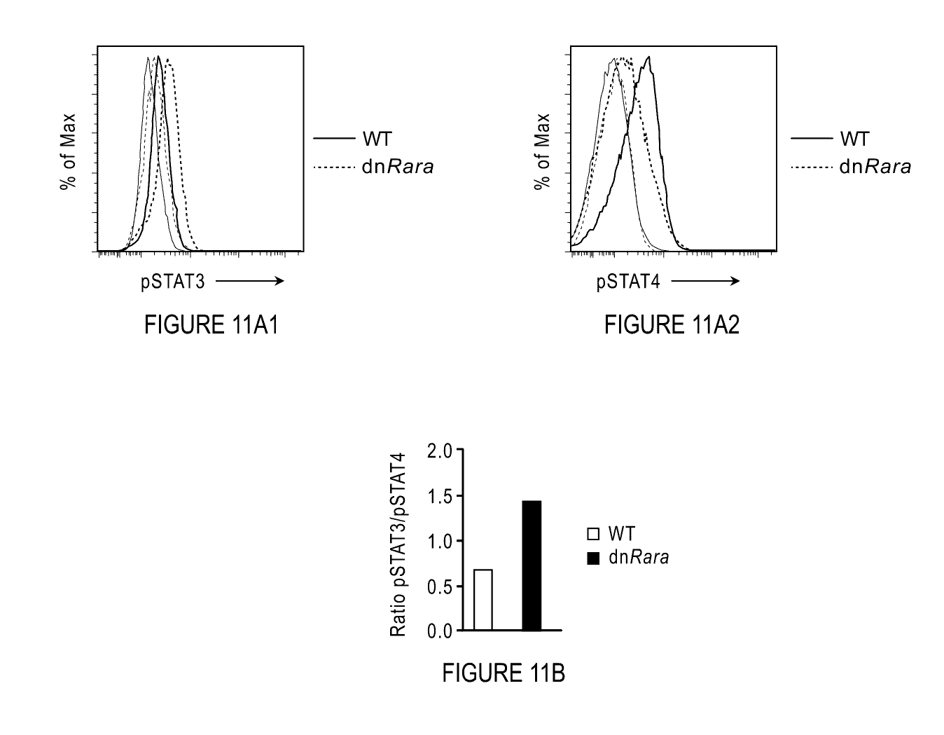

The late IL-12-dependent peak of T-bet expression observed in the presence of blocking IFN-γ antibodies was abrogated in dnRara Th1 cell polarised cells (FIG. 3A1-3A2) suggesting impaired STAT4 activity. At day 3 of culture, comparable amounts of phosphorylated STAT4 (pSTAT4) were observed between dnRara and WT mice. By contrast, at day 6 of culture, IL-12 induced pSTAT4 was markedly impaired in dnRara T-cells (FIG. 3B) despite comparable expression of IL-12Rβ2 mRNA and protein expression and increased expression of Il2rb1 mRNA Analysis of Stat4 expression, demonstrated impaired induction of Stat4 in the absence of RA signaling with reduced amounts of total STAT4 protein. These findings suggest that the observed reduction in pSTAT4 in dnRara Th1 cells is a consequence of diminished STAT4 expression. Consistent with deviation towards the Th17 cell lineage, we observed enhanced pSTAT3 activity in Th1 cell polarised dnRara cells with an increased ratio of pSTAT3/pSTAT4 (FIG. 11A-11A2).

FIGS. 11A1-B show enhanced pSTAT3 activity in Th1 cell polarised dnRara cells with an increased ratio of pSTAT3/pSTAT4.

To evaluate whether the impairment in T-bet and STAT4 expression correlated with changes in IFN-γ, the time-course of IFN-γ expression following initiation of Th1 cell polarisation was analysed in naïve dnRara-Ifng^{eYFP} expressing CD4⁺ T-cells. The kinetics of IFN-γ induction, as measured by frequency of eYFP⁺ cells, closely mirrored WT-cells during the first 72 hours of culture but expression was not sustained in the absence of RA signaling (FIG. 3G). Collectively these data show that RA plays a temporal role in Th1 differentiation, maintaining Th1 cell commitment through regulation of T-bet and STAT4.

### Example 4

### RA-RARα Regulates Th1 Cell Plasticity

Alterations in the stable expression of lineage-determining TFs are thought to underlie Th cell stability or plasticity. The emergence of Th1-Th17 cells together with the loss of T-bet expression, suggested a role for RA in the regulation of Th1 cell plasticity. However, diminished T-bet and STAT4 activity from day 3 of primary Th1 cell differentiation prevented assessment of lineage stability in fully differentiated Th1 cells. To determine whether RA-RARα was required for long-term Th1 cell fate, we differentiated naïve CD4+ T-cells from dnRara^{lsl/lsl} mice under Th1 cell conditions, treated them with TAT-Cre (Wadia et al., 2004) on days 5 and 7 and restimulated them under Th1 cell conditions for a further 5 days.

The treatment conditions with TAT-Cre were as follows. Sort purified naïve CD4+ T-cells were differentiated under Th1 conditions. After 5 days, cells were washed twice in serum free medium prior to treatment with 50 μg/ml TAT-Cre (Millipore) or medium alone (mock treatment). Cells were incubated at 37° C. for 45 minutes. The reaction was quenched with medium containing 20% FBS followed by further washing. Cells were expanded for 2 days followed by retreatment with TAT-Cre or media as before. Cells were then restimulated under Th1 cell conditions for 3 days and expanded for a further 2 days prior to analysis.

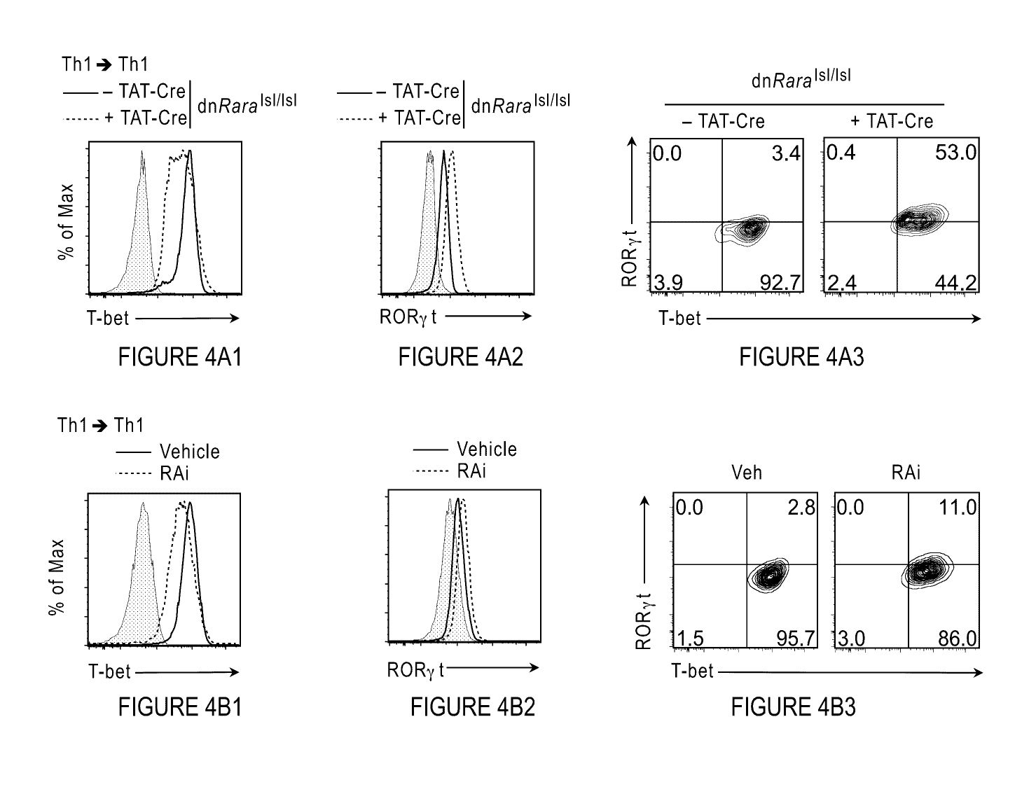

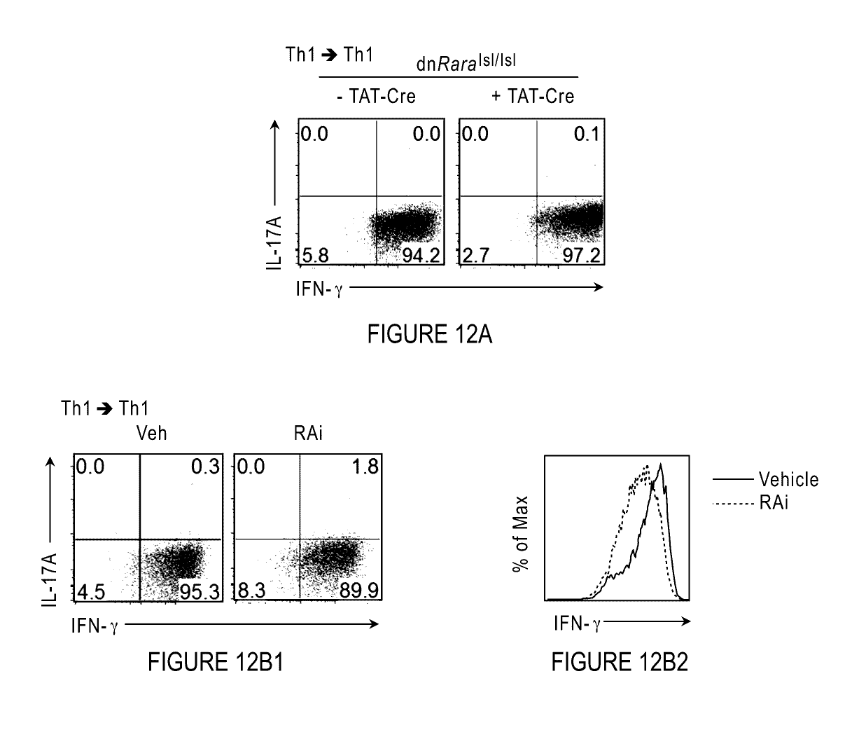

The temporal loss of RA signaling in Th1 cells resulted in decreased T-bet expression with a reciprocal increase in RORγt expression (FIG. 4A1-4A3). ˜50% of cells expressed RORγt, which suggests that ongoing RA-RARα activity is involved in sustaining T-bet and suppressing Th17 cell fate. Alterations in the lineage determining TFs did not impact on the cytokine phenotype (FIG. 12A). This may in part reflect T-bet independent regulation of the Ifng locus at late stages in Th1 cell development.

To further examine the role of RA in Th1 cell stability, naïve CD4⁺ T-cells from Ifng^{eYFP} mice were differentiated under Th1 cell polarising conditions. eYFP⁺ (IFN-γ⁺) cells were FACS-sorted on day 7 of culture and restimulated under Th1 cell conditions in the presence of the RAR inhibitor LE540 (RAi) or vehicle control (Veh). Inhibition of RA signaling in fully committed Th1 cells propagated for a further 5 days under Th1 conditions resulted in down-regulation of T-bet and the emergence of cells co-expressing RORγt (FIG. 4B1-4B3). Diminished T-bet expression was associated with modest reductions in IFN-γ expression (FIG. 12B1-12B2). Taken together these data establish that loss of RA signaling in fully committed Th1 cells leads to transdifferentiation to progeny with features of the Th17 lineage and support a model where RA constrains late stage plasticity of Th1 cells.

### Example 5

### RA-RARα Regulates Enhancer Activity at Lineage Determining Th1 Cell Genes

To better understand the molecular mechanism by which RARα regulates Th cell fate, we performed genome wide analysis of RARα binding in WT Th1 cells by ChIP-Seq, combined with transcriptional profiling of dnRara expressing Th1 cells in order to identify functional targets of RARα.

Immunoprecipitation and DNA sequencing was performed by Active Motif (Carlsbad, Calif.). The following antibodies were used: anti-H3K27me3 (Millipore 07-449), anti-p300 (Santa Cruz sc-551X), anti-H3K4me1 (Active Motif 39287), anti-H3K4me3 (Active Motive 39159), anti-H3K27ac (active Motif 39133), anti-RARα (Diagenode C15310155). Illumina sequencing libraries were prepared from the ChIP and Input DNAs. For ChIP q-PCR, enrichment calculated as binding events per 1000 Cells using Active Motifs normalisation scheme.

The experimental procedures were as follows. 20-60 million Th1 polarised cells from WT and dnRara mice were fixed, washed and snap-frozen according to the Cell Fixation protocol from Active Motif (www.activemotif.com/documents/1848.pdf). Chromatin was isolated by the addition of lysis buffer, followed by disruption with a Dounce homogenizer. Lysates were sonicated and the DNA sheared to an average length of 300-500 bp. Genomic DNA (Input) was prepared by treating aliquots of chromatin with RNase, proteinase K and heat for de-crosslinking, followed by ethanol precipitation. Pellets were resuspended and the resulting DNA was quantified on a NanoDrop spectrophotometer. Extrapolation to the original chromatin volume allowed quantitation of the total chromatin yield. An aliquot of chromatin was precleared with protein A agarose beads (Invitrogen). Following immunoprecipitation with specified antibodies, complexes were washed, eluted from the beads with SDS buffer, and subjected to RNase and proteinase K treatmnent. Crosslinks were reversed by incubation overnight at 65° C., and ChIP DNA was purified by phenol-chloroform extraction and ethanol precipitation and used for the preparation of Illumina sequencing libraries and for ChIP qPCR analysis.

A. ChIP-qPCR

Quantitative PCR (qPCR) reactions were carried out in triplicate on specific genomic regions using SYBR Green Supermix (Bio-Rad). See Table 3 for Primer details. The resulting signals were normalized for primer efficiency by carrying out qPCR for each primer pair using Input DNA. By using standards of known quantities of DNA it was possible to calculate the number of genome copies pulled down for each of the sites tested, and thus to calculate the copies pulled down per starting cell number, presented as ‘Enrichment’. For RARα ChIP qPCR a gene desert on chromosome 6 (Untr6) was used for a negative control site (Active Motif Catalog No: 71011).

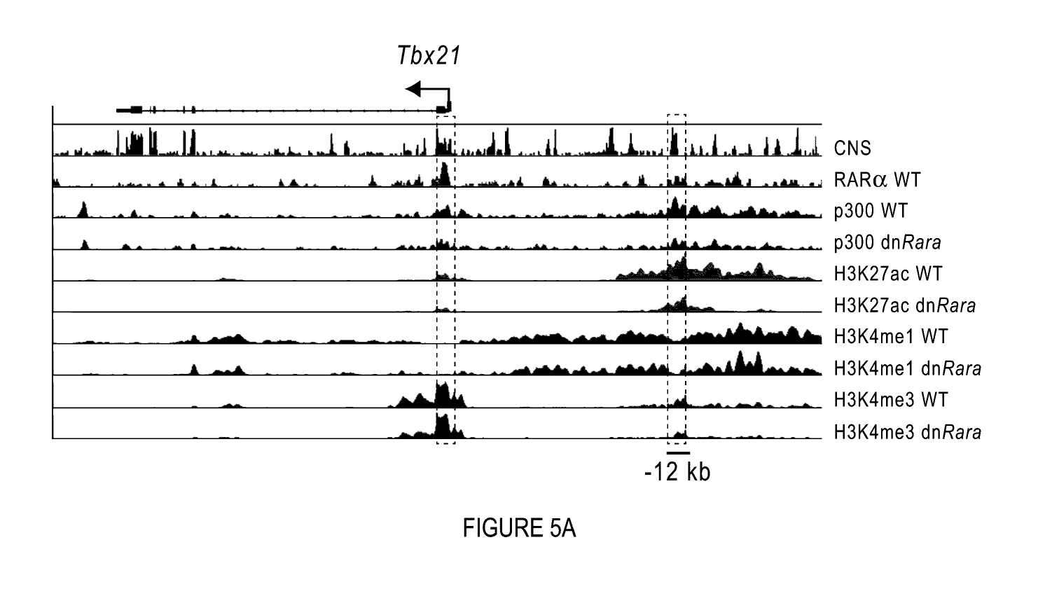

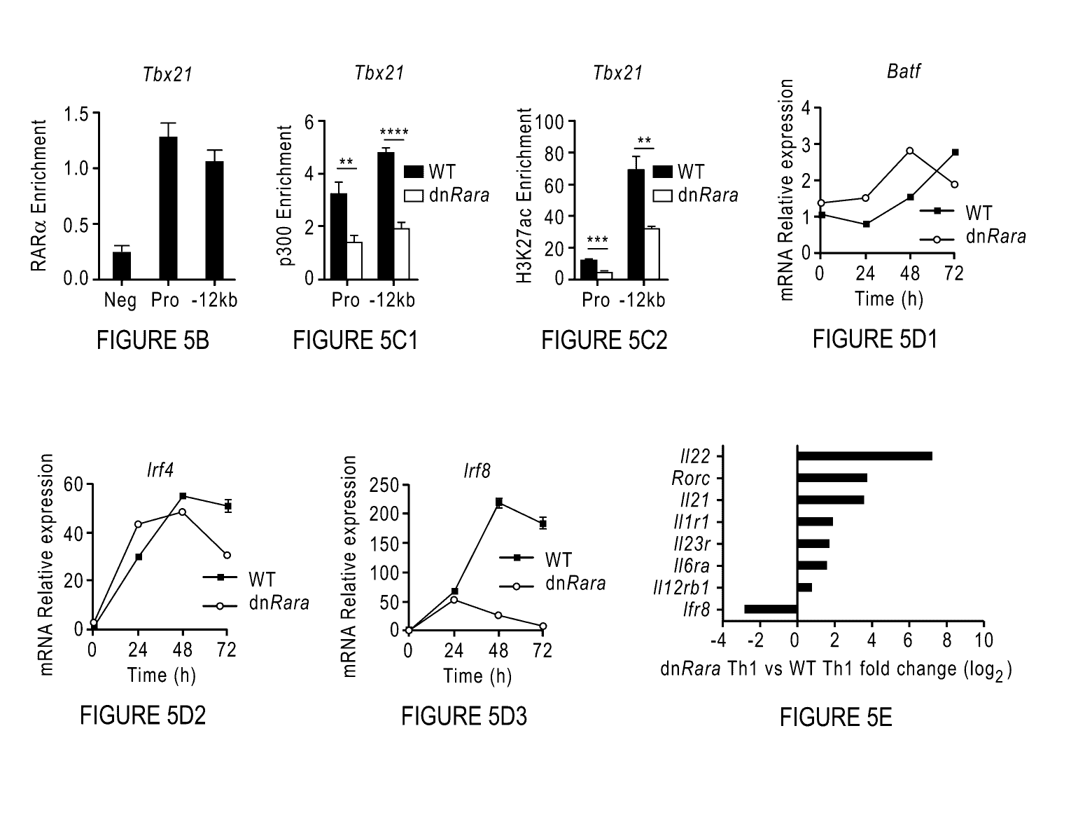

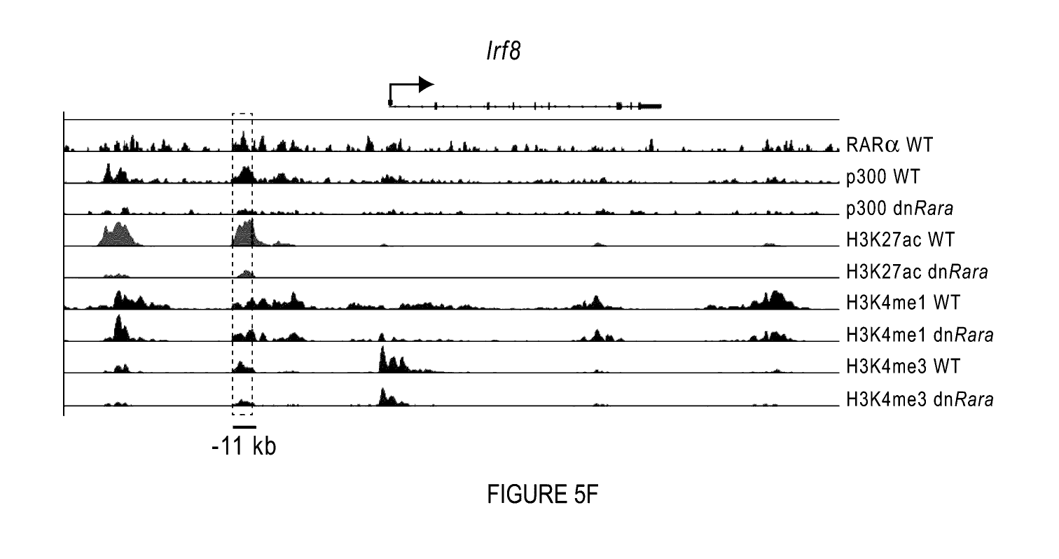

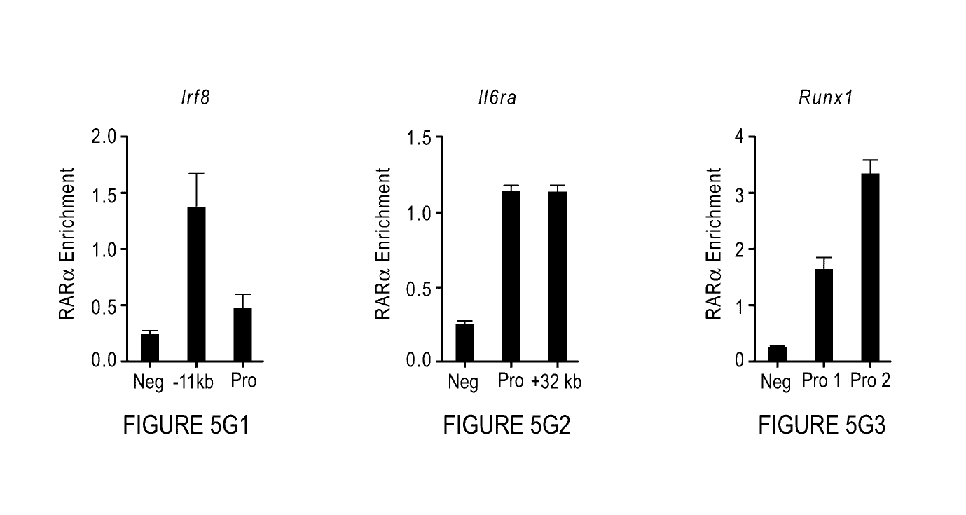

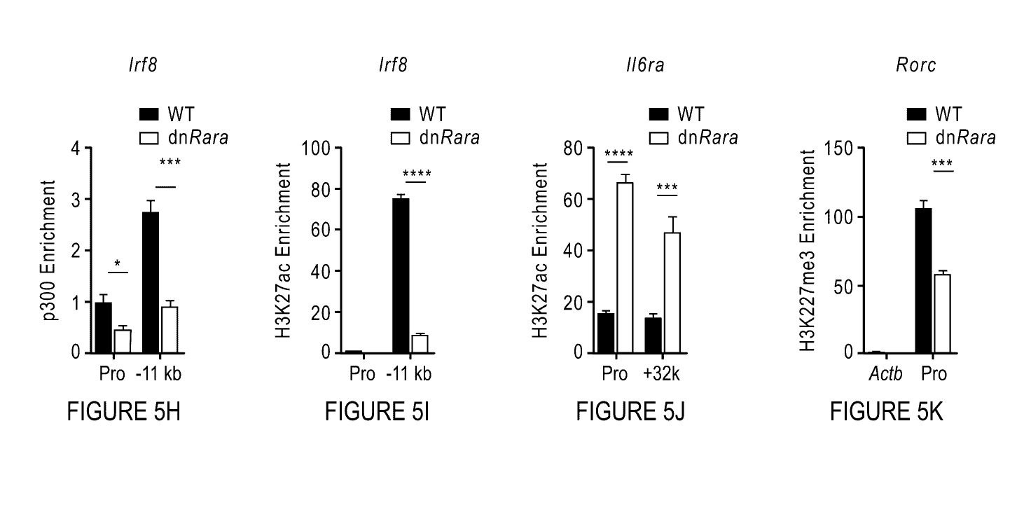

TABLE 3
(related to FIG. 5A-5K). Sequences of PCR
primers used in ChIP assays

| | | SEQ |
|---|---|---|
| | | ID |
| Description | Sequence | NO |
| Stat4_ + 105k F | TCCTCCTCCCTTTGTTGTTC | 1 |
| Stat4 + 105k R | GGGCCTTAATCAACCATTTC | 2 |
| Stat4 Promoter F | AGAGGGCATACACCGAGAAC | 3 |
| Stat4 Promoter R | TCTAGGGAGCCAGCATCAAC | 4 |
| Tbx21 Promoter F | TCGCTTTTGGTGAGGACTG | 5 |
| Tbx21 Promoter R | GGTGGCAGGTTGACTCTTTC | 6 |
| Tbx21 -12k F | GCGGAAGAGGGAACTAACAC | 7 |
| Tbx21 -12k R | GGACCCGGAACCTATGTATG | 8 |
| Irf8 Promoter F | CAGAAGCTAGGGCTGGTGTC | 9 |
| Irf8 Promoter R | CACAGAACAGATCCCAAATGTC | 10 |
| Irf8 -11k F | CCTTAACCCCGGAACTGTAG | 11 |
| Irf8 -11k R | TGCTGTGCTTGCCTCTACTC | 12 |
| Il6ra Promoter F | TCCGCTTGAGTTTTGCTTTC | 13 |
| Il6ra Promoter R | CACTGACCTGCCTTCTACTTTAAC | 14 |
| Il6ra + 32k F | CAAAGCTAAAACCAGGAAATGAC | 15 |
| Il6ra + 32k R | AAAAGGTTCCATGTGATGTTG | 16 |
| Rorc Promoter | AGGAATTTGGGTGTGGTGAG | 17 |
| (Rorγt isoform) F | | |
| Rorc Promoter | CTGTCTTGGGTGGTGTCTTG | 18 |
| (Rorγt isoform) R | | |
| Runx1 Promoter 1 F | TGGAAGAGGAAGAAGCTGTG | 19 |
| Runx1 Promoter 1 R | CAAGAGAAGCCACCCCAAAC | 20 |
| Runx1 Promoter 2 F | TGCTGGGCTTACACTTCTGAC | 21 |
| Runx1 Promoter 2 R | TGGACCTCATAAACAACCACAG | 22 |
| IFNg + 28k F | CTTTGAGCCACTGATGGGTAG | 23 |
| IFNg + 28k R | GCCTCTCCACGTCTCTTCTTC | 24 |

B. ChIP Sequencing (Illumina)

Illumina sequencing libraries were prepared from the ChIP and Input DNAs using standard procedures and libraries were sequenced on HiSeq 2500. ChIP-seq and microarray data are available under GEO accession number GSE60356.

C. ChipSeq Analysis

For each sample the 50 bp SE reads in FastQ format from the sequencer were aligned to the mouse reference genome (mm10) using Novoalign v2.07.11 (http://www.novocraft.com). The resulting alignment file was converted to BAM format using samtools (http://samtools.sourceforge.net/) and the PCR duplicates were removed using picard tools (http://picard.sourceforge.net). Only uniquely mapped reads from each sample were selected for further analysis. Significantly enriched regions from each sample were identified with MALCS v2.0.10_20131216 (Zhang et al. 2008, Feng J et al. 2011) (with q=0.10) using the input sample for background correction. In some instances, peaks were identified by visual inspection and confirmed by ChIP qPCR. In case of H3K4me1 and H3K27me3 samples, “-broad” setting was used to merge nearby enriched regions. For visualization purposes, the input signal was subtracted from each ChIP sample and was converted into bigWig format using “bedGraphToBigWig” utility from UCSC tools (http://genome.ucsc.edu/util.html). The identified significantly enriched regions were annotated to find the associated genes using “FindNeighbouringGenes” utility from USeq package (useq.sourceforge.net/). Associated genes represent the closest transcriptional start site from the centre of the peak.

D. ChipSeq Results

Selected loci were validated by ChIP-qPCR. RARα binding was identified at 1766 sites in 1567 genes. RARα binding was detected at 10.3% (76 of 740 genes) of genes down-regulated in the absence of RA signaling (Table 4) (hereafter referred to as positively regulated) and 4.8% (56 of 1169) of the up-regulated genes (Table 5). In keeping with its classical role as a positive regulator of transcriptional activation there was significant enrichment of RARα binding at genes positively regulated by RA (Fisher exact test, p<0.0001). However, the presence of RARα at a subset of the negatively regulated genes indicates that RA-RARα also plays a role in transcriptional repression within Th1 cells.

TABLE 4
(related to FIG. 5A-5K). Genes downregulated in dnRara
Th1 cells that were bound by RARα in WT Th1 cells

1110037F02Rik
1810011H11Rik
3300005D01Rik
5830416P10Rik
Acsl4
Adora2a
Alkbh7
Asb2
Birc5
Blm
Bre
Capzb
Chsy1
Cmas
Cnga1
Coq7
Ctps
Cycs
Cyp51
Cyp51
Dennd4a
Dusp6
E2f3
Enpp4
Fasn
Fgl2
Fli1
Fmnl3
Foxo3
Foxp1
Furin
Gas5
Gcsh
Gfi1
Gimap3
Gimap4
Gimap8
Gimap9
Hic1
Hmgcs1
Idi1
Ifngr1
Ifrd2
Irf8
Itih5
Kcnn4
Kif2c
Lbr
Lef1
Mdc1
Me2
Mrto4
Ncln
Nedd4l
Nfic
Nln
Nme1
Nod1
Notch2
Nt5e
P2rx7
Pde2a
Prr5l
Rbks
Rcbtb2
Shf
Slc16a6
Smad3
Sqle
Sulf2
Tbx21
Treml2
Txn2
Ube2e3
Uchl3
Vav3
Vipr1

TABLE 5
(related to FIG. 5A-5K). Genes upregulated in dnRara
Th1 cells that were bound by RARα in WT Th1 cells

1110038F14Rik
Ak2
Antxr2
Aph1b
Arhgap25
Arid4a
B2m
Bace2
Bcl10
Bcl6
Birc3
Cd320
Cnnm2
Ddit3
Egr2
Fam43a
Filip1l
Fndc3a
Fuca1
Ifngr2
Il15ra
Insr
Irf1
Irgm1
Kif3b
Mcl1
Mettl8
Mga
Mpeg1
Nek6
Net1
Npc2
Plec
Polg
Ptpn1
Rab19
Rhd
Slamf1
Slfn2
Socs1
Sp100
Stat1
Tagap
Tmem50a
Tnip1
Tor1aip2
Traf1
Trpm6
Twsg1
Usp53
Vav1
Wdsub1
Zbp1
Zfp207
Zfp36l2
Zmym6

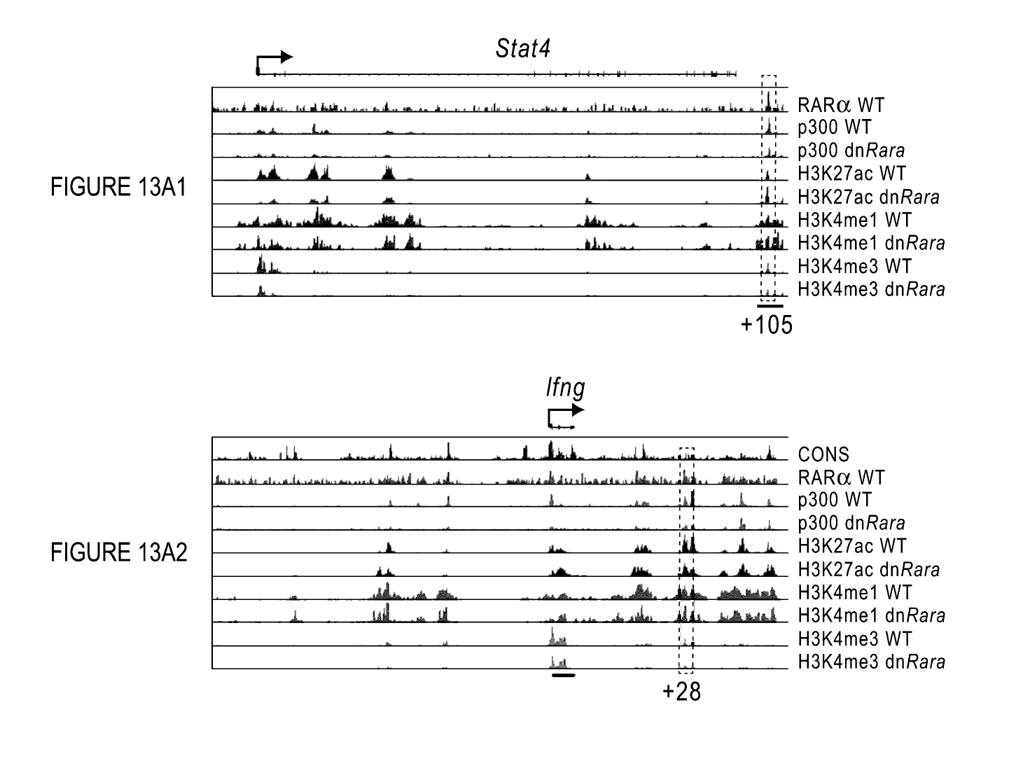

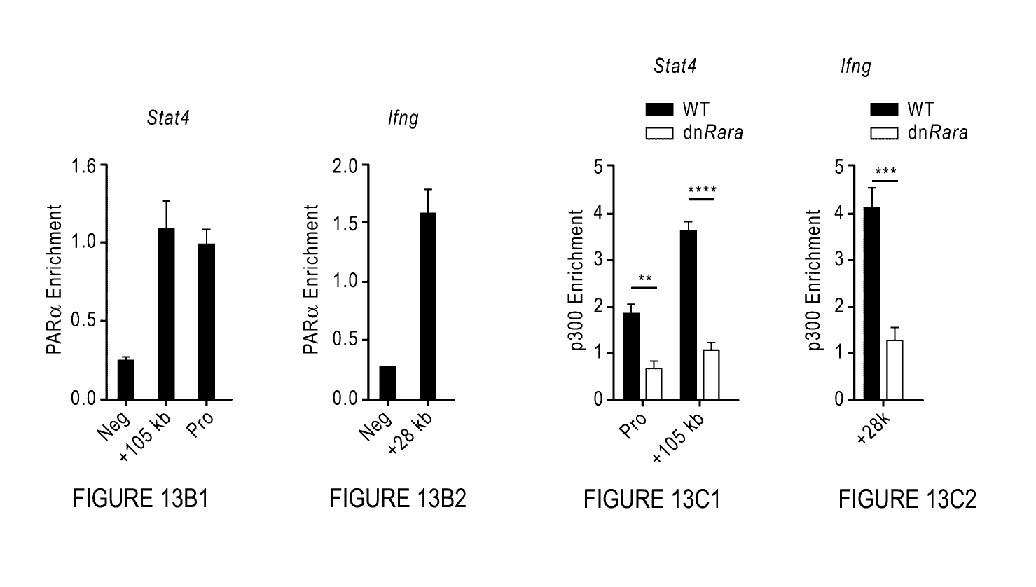

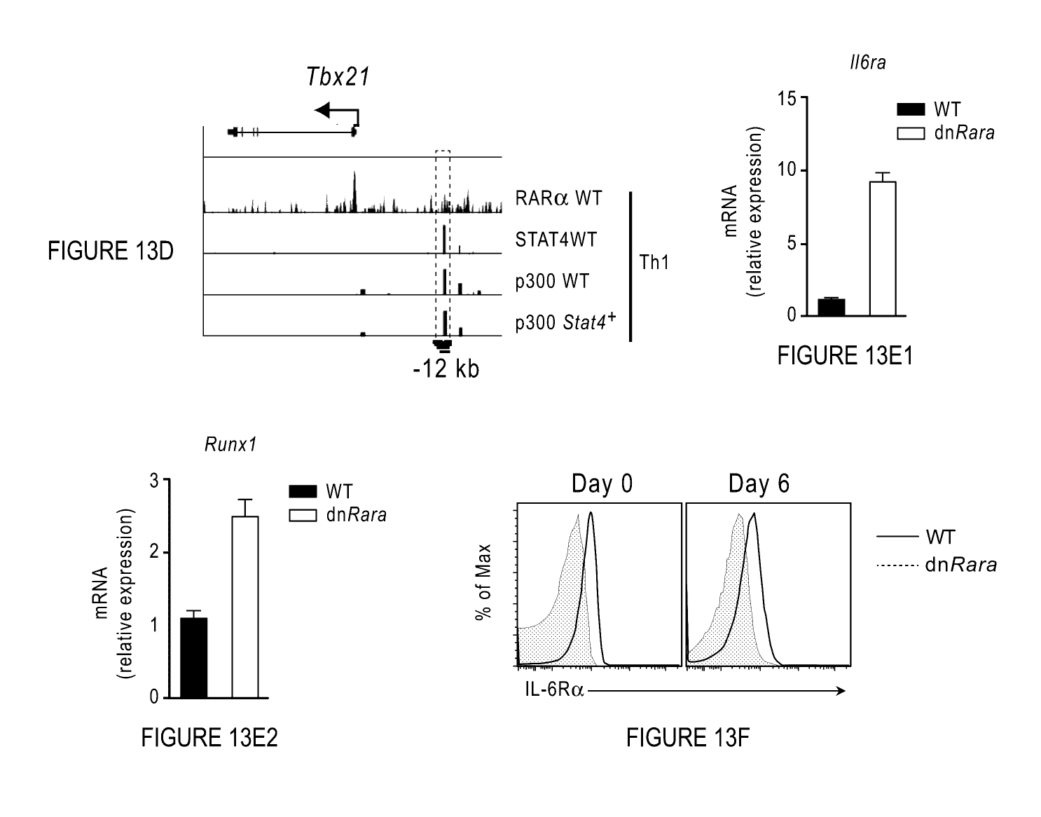

RA-RARα dependent loci included Th1 cell lineage-defining genes (Tbx21 and Stat4-Stat1). In addition to targeting the Tbx21 promoter (FIG. 5A and FIG. 5C1-5C2), modest RARα binding was observed at the conserved T-bet enhancer element, 12 kb upstream of the transcriptional start site (TSS) (Yang et al., 2007). This was confirmed by ChIP-qPCR (FIG. 5C1-5C2). Intergenic RARα was also detected at the Stat4-Stat1 locus and an Ifng enhancer element (FIG. 13A1-13B2).

RA binding to nuclear RARα results in recruitment of co-activator complexes containing the histone acetyl-transferases p300 and CBP (Kamei et al., 1996). p300 is highly enriched at enhancer regions where it acetylates H3K27, a marker of active enhancers (Rada-Iglesias et al., 2010), suggesting a possible role for RA-RARα in regulating enhancer activity. To test this, we mapped genome wide binding of p300, H3K4me1, H3K4me3 and H3K27ac histone modifications in dnRara and WT Th1 cells, validating selected regions by ChIP q-PCR. Active enhancers were operationally defined as regions with increased intensity of H3K4me1, p300 and H3K27ac with low or absent H3K4me3 (Rada-Iglesias et al., 2010).

RARα binding at the Tbx21, Stat4 and Ifng loci co-localised with p300 binding at enhancer regions (FIGS. 5A and 13A1-13A2). dnRARα lacks the activation function 2 (AF2) domain which is required for RA-dependent recruitment of coactivators. Consistent with this, dnRara expressing T-cells exhibited a significant reduction in p300 occupancy and H3K27ac deposition at the Tbx21 enhancer, supporting the direct regulation of enhancer activity by RA-RARα(FIGS. 5A and 5C1-5C2). p300 binding at the Ifng and putative Stat4 intergenic enhancers was also dependent on RA-RARα (FIGS. 13A1-13A2 and 13C1-13C2). Loss of p300 binding at the Stat4-Stat1 intergenic enhancer in dnRara Th1 cells correlated with reduced Stat4 transcripts whereas Stat1 expression was actually increased, suggesting that this enhancer element regulated Stat4 transcription. A recent study identified a role for STAT4 in the regulation of Th1 enhancers (Vahedi et al., 2012). Given that STAT4 expression was reduced in dnRara Th1 cells, it was possible that the loss of p300 was in part due to reduced expression of STAT4. To address this issue we assessed the binding of STAT4 in WT Th1 cells and compared p300 occupancy in WT and Stat4^{-/-} Th1 cells using publically available ChIP-seq data (Table 6) (Vahedi et al., 2012; Wei et al., 2010). Although STAT4 binding was observed at the Tbx21 enhancer, loss of STAT4 was not associated with obvious differences in p300 binding (FIG. 13D) arguing for a direct contribution of RARα to p300 recruitment and enhancer activity. Collectively these data show that RA regulates expression of certain Th1 cell lineage genes through remodeling of enhancer regions.

TABLE 6
(related to FIG. 5C1-5C2). List of Sequencing-Based
Data Used in This Study including publically available
data as indicated by Geo Accession Number

| Samples | Non-redundant tags | Peak counts |
|---|---|---|
| RARA_WT | 13303876 | 1776 |
| H3K4me1_DNRAR | 18605274 | 65960 |
| H3K4me1_WT | 23760603 | 49542 |
| H3K4me3_DNRAR | 18333386 | 49505 |
| H3K4me3_WT | 21918629 | 53135 |
| H3K27Ac_DNRAR | 17421600 | 37788 |
| H3K27Ac_WT | 20513640 | 37151 |
| H3K27me3_DNRAR | 30667883 | 56002 |
| H3K27me3_WT | 20833021 | 78511 |
| p300_DNRAR | 23023765 | 30495 |
| p300_WT | 25213927 | 46191 |
| Stat4 WTTh1 (GSM550303) | 8982352 | 20862 |
| p300 WT Th1 (GSM994508) | 19652779 | 25554 |
| p300 Stat4^{-/-} Th1 | 18282554 | 29208 |
| (GSM994509) | | |

### Example 6

### RA-RARα Represses Th17 Cell Fate in Th1 Cells Through Direct Regulation of Th17 Cell Genes

The earlier finding that Th1 cells acquired features of Th17 cells in the absence of RA signaling led us to evaluate direct regulation of Th17 cell instructing genes by RA-RARα. We first investigated effects of RA on the Th17 cell pioneer factors BATF and IRF4. As previously reported (Basu et al., 2013), these genes were expressed in WT Th1 cells. Strikingly, kinetic analysis of Batf and Irf4 expression in naïve cells stimulated under Th1 cell conditions revealed dramatic up-regulation of IRF4 (40- to 60-fold) during the initial phase of Th1 cell polarisation with comparable expression between dnRara and WT-cells (FIG. 5D1-5D3). Loss of RA signaling resulted in derepression of BATF-IRF4 target genes, Rorc, Il23r, Il22, Il21 and Il12rb1 (FIG. 5E). This suggested that ‘balancing’ factors must be induced in an RA dependent manner to restrict the actions of BATF-IRF4 complexes at Th17 cell genes. IRF8, an alternative binding partner for IRF4, previously shown to suppress Th17 differentiation (Ouyang et al., 2011), was one of the RARα target genes most suppressed in dnRara Th1 cells. In WT Th1 cells, induction of Ir8 expression paralleled Irf4 expression. However, in dnRara cells Irf8 expression was not sustained past 24 h (FIG. 5D1-5D3). RARα bound at a putative upstream enhancer (FIG. 5F-5G3) and in the absence of RA signaling, reduced p300 and H3K27ac were observed at this locus (FIG. 5H-I). Together these data show that RA directly regulates expression of IRF8 in Th1 differentiating cells and suggests a potential mechanism by which BATF-IRF4 activity is constrained within early Th1 cells.

Transcriptional activation of BATF-IRF4 target genes is dependent on STAT3 and RORγt (Ciofani et al., 2012). Various genes for cytokines and cytokine receptors associated with STAT3 activation (Il21, Il1r1, Il6ra and Il23r) were derepressed in dnRara Th1 cells (FIG. 5E). RARα targeted the promoter and an upstream enhancer in the Il6ra locus (FIG. 5G1-3) with increased H3k27ac observed at the enhancer element in dnRara Th1 cells (FIG. 5J). Consistent with this, dnRara Th1 cells failed to down regulate mRNA and cell surface IL6-Rα expression during Th1 polarisation (FIGS. 13E1-2 and 13F). These findings suggest that RA regulates Th1 cell plasticity in part by inhibiting responsiveness to IL-6.

RORγt was not a direct target of RARα. However, disruption of RA signaling resulted in increased expression of Runx1, a TF associated with transactivation of Rorc (FIG. 13E1-13E2) (Zhang et al., 2008). ChIP analysis confirmed direct regulation of short and long Runx1 isoform promoters by RA-RARα (FIG. 5G1-5G3). In Th1 cells, the Rorc locus is epigenetically silenced by T-bet (Mukasa et al., 2010). However, in dnRara cells, the repressive H3K27me3 mark was reduced at RORγt isoform specific exon (FIG. 5J), consistent with loss of T-bet. These findings suggest that increased RORγt expression in the absence of RARα signaling is in part due to increased accessibility of the Rorc locus, with unrestrained activation by Runx1. Collectively these data indicate that RA-RARα antagonises the activity of the core Th17 cell instructing TFs (IRF4, BATF, STAT3 and RORγt), both directly and indirectly, to suppress the Th17 cell gene program. Notably, Th2 cell-associated genes were not identified as targets of RARα (Table 5 and 4) suggesting that direct repression of alternative cell fates by RA-RARα is specific to the Th17 cell program.

### Example 7

### Th1-like Th17 Cells Emerge During Infection with L. monocytogenes in the Absence of RA Signaling

To assess the significance of these findings for immune responses in vivo, WT and dnRara mice were infected intravenously with an attenuated strain of L. monocytogenes (ΔActA), Lm-2W, which allows tracking of CD4⁺ T-cells specific for listeriolysin O peptide LLO₁₉₀₋₂₀₁ (LLOp).

LLO₁₉₀₋₂₀₁ was synthesised by PiProteomics and was >95% pure, as determined by HPLC. LLO:I-A^{b} monomers were provided by NIH Core Tetramer Facility. PE labeled LLO:I-A^{b} dextraners were synthesised by Immudex. Recombinant Lm-2W strain was provided by Marc Jenkin's Laboratory. LE540 was purchased from Alpha Laboratories.

Mice were infected i.v. with 1×10⁶ cfu L. monocytogenes and spleens were harvested 7 days later. For FACS analysis, single cell suspensions were enriched for CD4⁺ T-cells with a CD4⁺ T-cell negative selection microbead kit (Miltenyi Biotec) and stained with PE labeled, LLO:I-A^{b} dextramer (Immudex) and cell surface antibodies. For analysis of cytokine production, supernatants were collected from splenocytes restimulated with LLO peptide (PiProteomics) at 10 μg/ml for 24 h or intracellular cytokine staining was performed following stimulation with LLO peptide for 6 h in the presence of monensin.

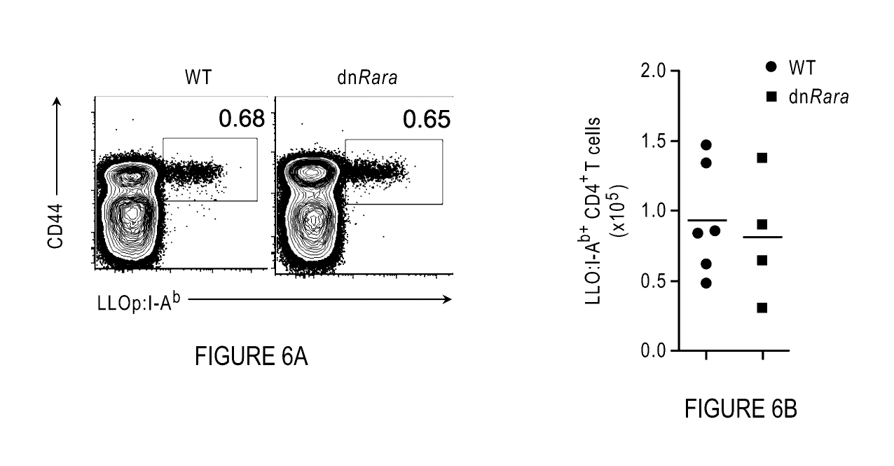

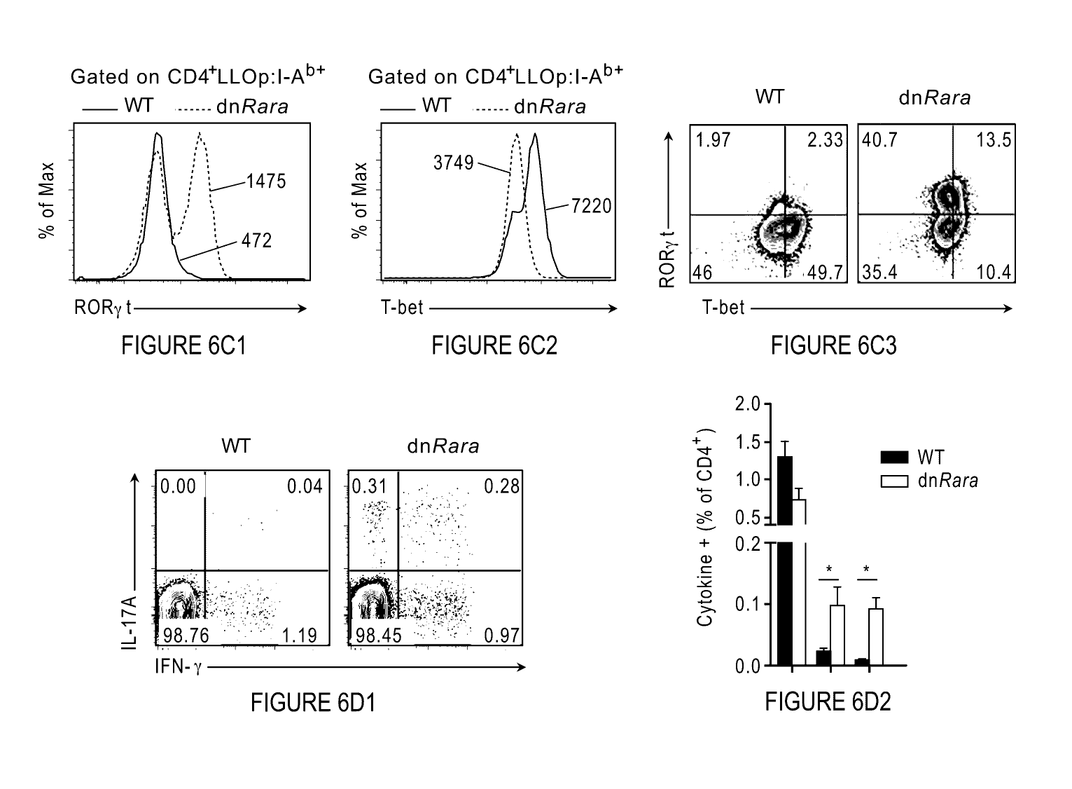

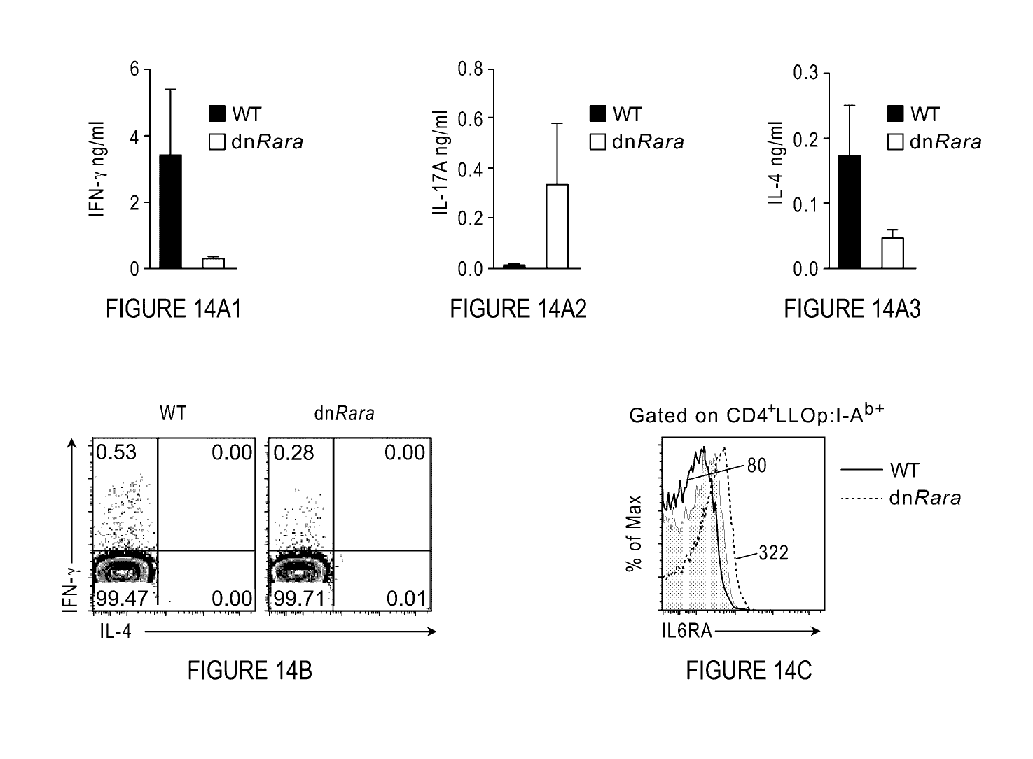

At the peak of the response, CD4⁺ T-cells were isolated from the spleen and LLOp antigen specific T-cells were assayed for expression of cytokines and the TFs, T-bet and RORγt. dnRara mice mounted an effector T-cell response of similar magnitude to WT mice with comparable frequencies and total numbers of CD44^{hi}LLOp:I-A^{b}-specific CD4⁺ T-cells (FIG. 6A-B). In WT mice, Lm-2W induced a Th1 cell restricted response, as evidenced by high T-bet expression within the LLOp specific T-cell fraction (FIG. 6C1-6C3). LLOp:I-A^{b+} CD4+ T-cells from dnRara mice expressed lower amounts of T-bet and a substantial proportion expressed RORγt, with co-expression of these TFs observed in a subset of cells (FIG. 6C1-6C3). At day 7 post-infection, a significant proportion of CD4⁺ T-cells isolated from the spleen of dnRara mice were IL-17⁺ or dual IL-17A⁺IFN-γ⁺ with a trend towards reduced frequency of IFN-γ⁺ cells (FIG. 6D1-6D2). Measurement of cytokine protein concentrations from splenocytes restimulated with LLOp confirmed reduced amounts of IFN-γ and concomitant increase in IL-17A (FIG. 14A1-14A3). We did not detect IL-4 production by intracellular staining or protein secretion (FIG. 14A-B). Consistent with our in vitro data showing down-regulation of IL6-Rα on WT Th1 cells, cell surface IL-6Rα was not detectable on WT LLOp:I-A^{b+} CD4⁺ T-cells. However, dnRara LLOp:I-A^{b+}CD4⁺ T-cells retained expression of IL-6Rα (FIG. 14C), supporting a potential role for IL-6 signaling in the regulation of Th1 cell plasticity. These findings establish that RA-RARα signaling in T-cells constrains the emergence of Th17 cells in a Th1 cell instructing microenvironment in vivo.

### Example 8

### RA Regulates the Th1-Th17 Cell Axis in the Gut and Prevents the Development of Intestinal Inflammation

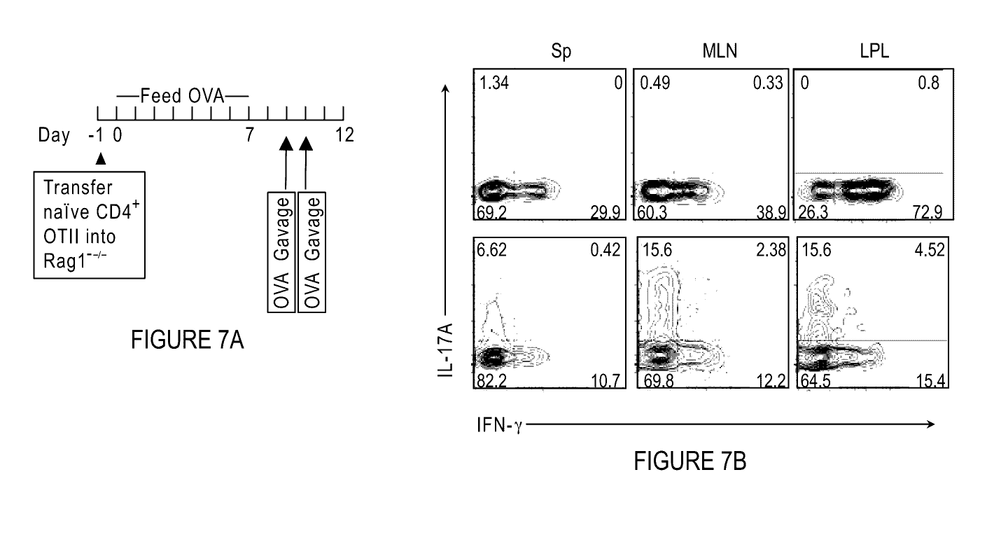

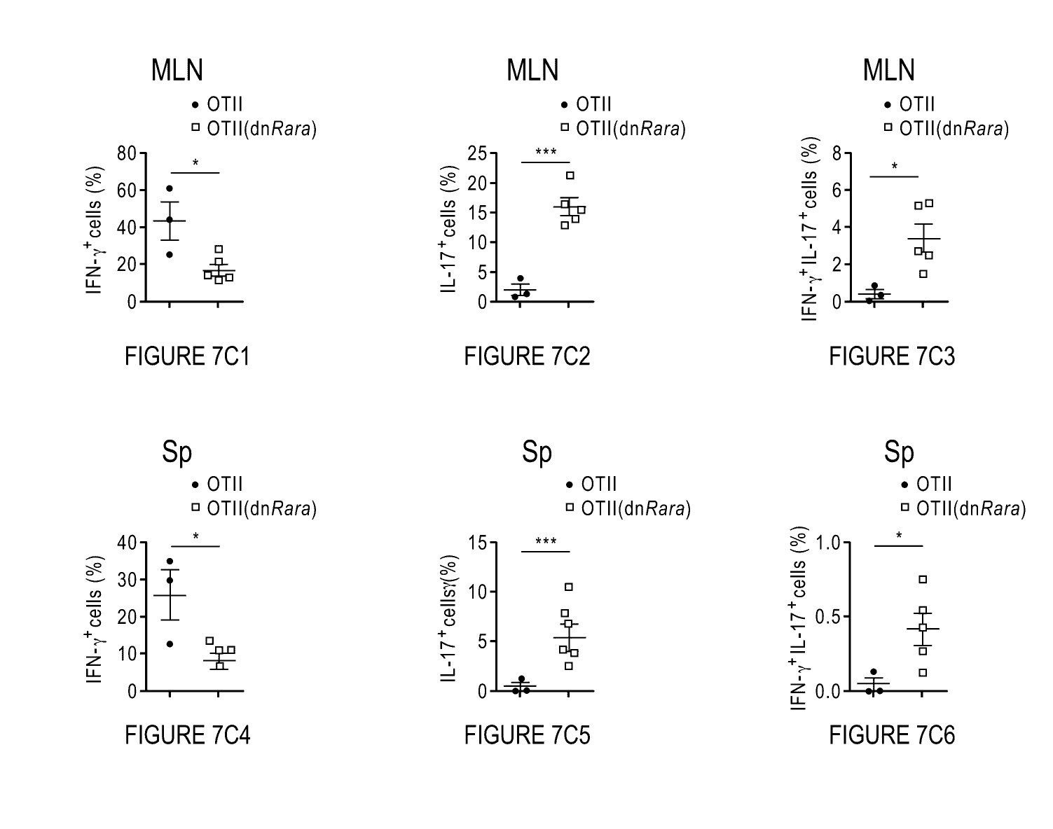

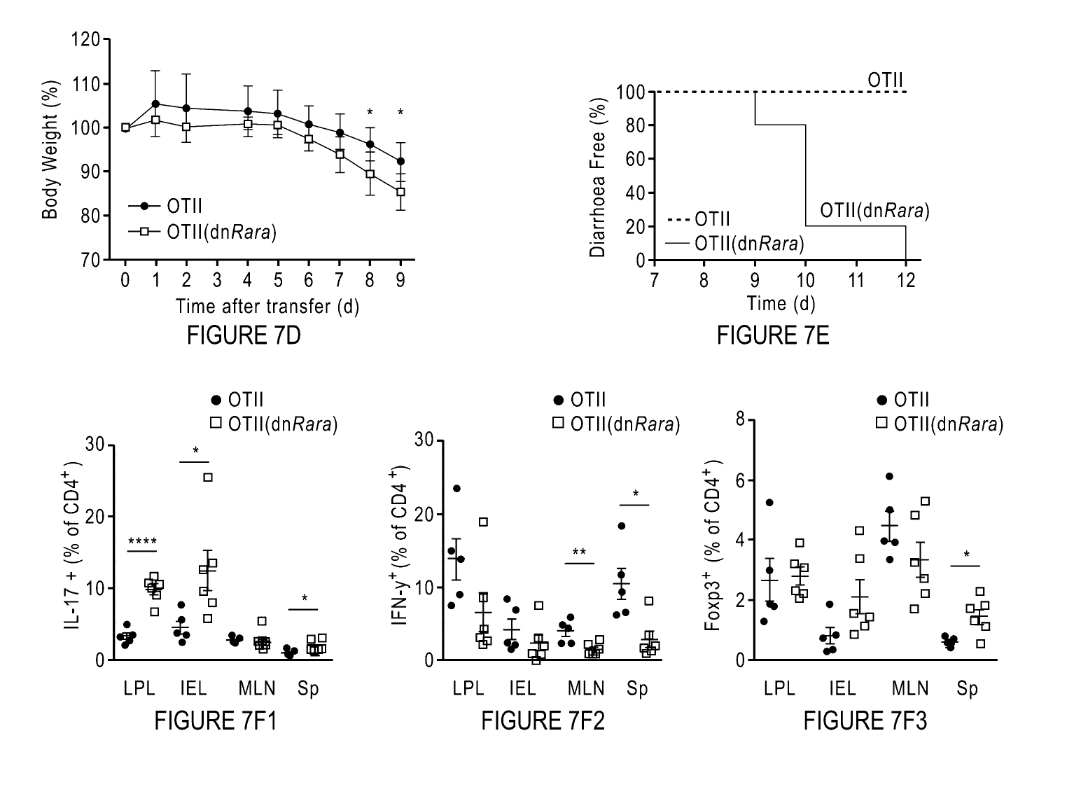

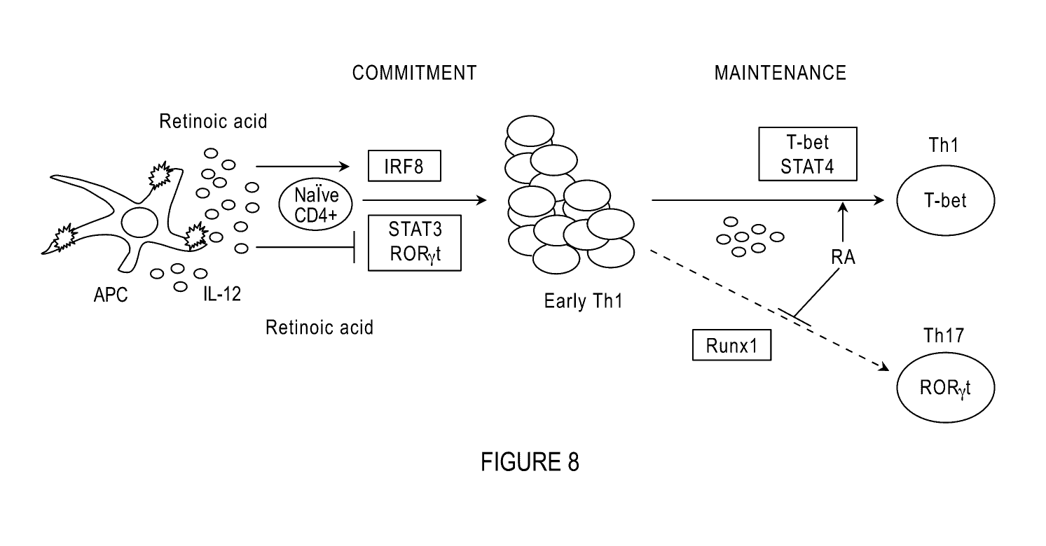

RA is constitutively synthesised by a subset of DCs in the gut. To address the physiological importance of RA signaling in the regulation of pathogenic intestinal CD4⁺ T-cells, we interbred dnRara mice with OTII mice that transgenically express an ovalbumin (OVA) specific TCR and transferred naïve CD4⁺ T-cells from OTII(dnRara) or WT OTII mice into Rag1^{-/-} hosts. C57Bl/6 OTII(dnRara), OTII and Rag1^{-/-} mice were bred and maintained at the Rockefeller University specific pathogen free animal facility. Recipients were maintained on an OVA-containing diet for 7 days to induce differentiation within the transferred cells and migration to the intestinal tissue. Consistent with the infection experiments, feeding OTII(dnRara) recipient mice OVA resulted in a shift in the Th1-Th17 cell balance with a deficiency in IFN-γ producing cells and increased frequency of IL-17⁺ and dual IFN-γ⁺IL-17⁺ cells in the mesenteric lymph node (MLN), lamina propria lymphocytes (LPL) and spleen (Sp), 7 days after transfer (FIGS. 7B and 7C1-7C6). To address the functional significance of the dysregulated cytokine response in dnRara T-cells, mice were orally challenged with OVA and evaluated for development of intestinal inflammation and diarrhoea (FIG. 7A).

Rag1^{-/-} mice were kept on a sulfatrim-containing diet and only exposed to autoclaved supplies. Naïve OTII CD4 cells (defined as CD4⁺CD25⁻Vb5⁺Va2⁺CD44⁻) were sorted from 8-12 weeks old female C57Bl6 OTII(dnRara) or C57Bl6 OTII mice using a FACS Aria cell sorter (Becton Dickinson), and 2×10⁶ cells in 100 μl PBS were retro-orbitally transferred to 12 weeks old Rag1^{-/-} females. 12 h after the adoptive transfer, the drinking water was replaced by a 1% Grade II ovalbumin (OVA, Sigma) and 0.5% Splenda (McNeil Nutritionals) solution for 7 days. Body weight was measured at 5 pm every day. For monitoring diarrhea development, the faeces texture after 7 days of OVA, 2 h after a gavage challenge with 50 mg Grade III OVA (Sigma) in 200 μl PBS on days 9 and 10 and without further challenge on day 12 was analysed. A mouse was diagnosed with diarrhoea if the faeces had the characteristic soft and light appearance at two consecutive occasions. For the single gavage challenge experiment, mice were subjected to the challenge on day 9 only and the faeces were analysed after 2 h. To determine T-cell frequencies, lymphocytes were isolated as previously described (Mucida et al., 2007) on day 7 (from mesenteric lymph node (MLN) and spleen only) or day 9 (from the intestinal epithelium, lamina propria, MLN and spleen) after the start of oral OVA exposure of the recipient mice. For cytokine staining, isolated lymphocytes were stimulated for 3 h in RPMI medium supplemented with 10% FBS, 55 μM β-mercaptoethanol, 100 ng/ml PMA (Sigma), 500 ng/ml Ionomycin (Sigma) and 10 μg/ml brefeldin A (Sigma) prior to the incubation with antibodies. Cells were first stained with antibodies against T-cell surface markers, followed by permeabilization using either Fix/Perm buffer (BD Pharmingen) for cytokine stainings, or using the Foxp3 Mouse Regulatory T-cell Staining Kit (eBioscience) for Foxp3 staining. The fluorescent-dye-conjugated antibodies used were obtained from BD-Pharmingen (anti-CD4, 550954; anti-CD25, 553866; anti-IL-17a, 559502; anti-Vb5, 553190) or eBioscience (anti-CD44, 56-0441; anti-CD45.2, 47-0454; anti-TCR-β, 47-5961; anti-IFN-γ, 25-7311; anti-Foxp3, 17-5773; anti-Vα2, 48-5812). Stained cells were analysed using a LSR-II flow cytometer (Becton Dickinson) and population frequencies were determined using the FlowJo software (Tree Star).

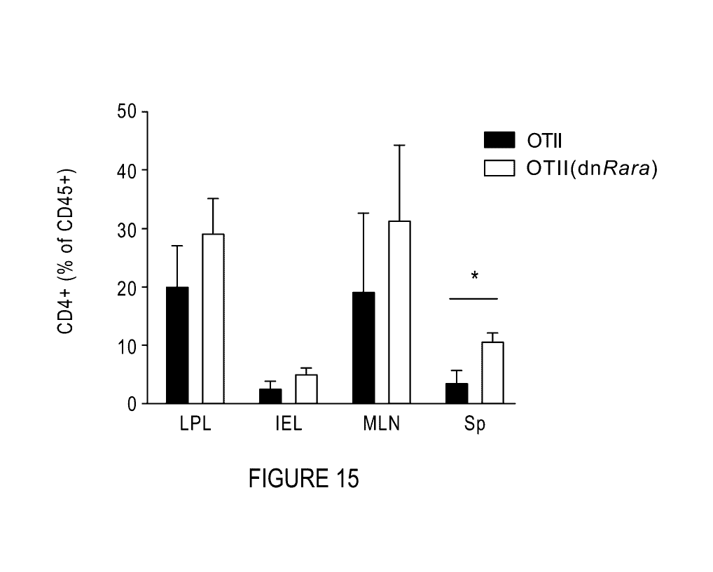

Recipients of OTII(dnRara) cells developed accelerated wasting disease relative to mice that received WT OTII cells (FIG. 7D). Whereas all of the recipients of OTII(dnRara) cells developed severe diarrhoea by day 12 (FIG. 7E), recipients of WT-cells remained diarrhoea free. Cytokine production was also assessed after the first gavage and confirmed an increased frequency of IL-17⁺ cells with concomitant reduction in IFN-γ⁺ cells. Notably, enhanced IL-17 responses were not a consequence of impaired Foxp3+ conversion (FIG. 7E). Homing of transferred cells to the gut was not affected in this model with similar frequencies of CD4⁺ T-cells detected in the gut tissues (FIG. 15). We conclude that loss of RA signaling leads to deviation from Th1 to Th17 phenotype both in the periphery and the gut where these Th17 cells are associated with significant intestinal inflammation.

### Example 9

### Discussion

Dysregulated Th cell responses underlie the pathogenesis of autoimmune and allergic disease. In contrast to T regulatory (Treg) cells and Th17 cells, the Th1 cell lineage is thought to be relatively stable. However, the factors that control maintenance of the Th1 cell lineage were not previously known. This study identifies RA-RARα as a central regulatory node in the transcriptional network governing Th1 cell stability. We found that RA-RARα directly sustained the expression of lineage determining Th1 cell-associated genes during naïve T-cell differentiation whilst also repressing signature Th17 cell-associated genes. Ablation of RA signaling in Th1 committed cells resulted in enhanced Th1 cell plasticity with deviation towards a Th17 cell phenotype. Using ChIP-seq to identify regulatory elements, we found that RARα bound at enhancers and recruitment of p300 to these regions was dependent on RA signaling. In vivo, both Th17 and Th1-Th17 cells emerged during infection with L. monocytogenes and in a model of oral tolerance. In the latter, their presence was associated with significant pathology.

Enhancers play a role in directing cell fate through the regulation of lineage specifying genes. Enhancer profiling in WT and dnRara T-cells revealed RA dependent activation of enhancers at genes involved in Th1 identity (Tbx21, Stat4, Ifng and Irf8). RA dependent changes in p300 and H3K27ac were reflected at the transcriptional level suggesting that, in addition to its classical role as a transcriptional regulator, RA regulates gene expression in an enhancer dependent manner. Although the ability of RA-RARα to target p300-CBP complexes to nucleosomes is well established, regulation of enhancers by RA has not been widely studied. We propose that unliganded RARα at enhancer elements acts as a gatekeeper, enabling initiation of enhancer activation once T-cells sense RA in the microenvironment. A similar role has been demonstrated for STAT proteins (Vahedi et al., 2012), suggesting that environmental cues act as checkpoints for initiation of enhancer activation and T-cell fate. Although H3K4me1 modifications are present at early timepoints during T-cell differentiation, conversion to ‘active’ status requires acquisition of H3K27ac, which is often not evident until later stages of differentiation (Larjo et al., 2013). Consistent with a temporal role for enhancers in maintenance of gene expression, RA signaling was not required for initiation of transcription of target genes but rather acted to maintain their expression. These data highlight the importance of enhancers in maintenance of cell identity and plasticity. It is possible that RA-RARα regulation of enhancers represent the major mechanism by which RA regulates cell fate. A recent study identified enrichment of RARα at enhancers in embryonic stem cells (Chen et al., 2012). Given that the RA-RARα axis is a highly conserved signaling pathway, which plays a role in regulating cell fate specification during embryogenesis and cell differentiation, it will be important to evaluate a broader role for RA-RARα in regulation of enhancer functionality, both in alternative Th cell subsets and outside of the immune system.

In addition to sustaining expression of Th1 cell-associated genes, we found that RA actively silences genes implicated in Th17 cell differentiation. Among genes known to regulate the Th17 cell program, Runx1 and Il6ra were directly repressed by RA-RARα. In addition, BATF-IRF4 target genes were derepressed in the absence of RA signaling. In Th17 cells, BATF-IRF4 complexes act co-operatively as pioneer factors at certain Th17 genes (Ciofani et al., 2012), modulating chromatin accessibility to facilitate binding of STAT3 and RORγt. Based on their expression in alternative Th cell subsets, it has been suggested that BATF-IRF4 complexes play a universal role in establishing binding of lineage specific TFs (Ciofani et al., 2012). However, BATF deficiency does not impact on Th1 cell differentiation (Schraml et al., 2009). An alternative model is that up-regulation of BATF and IRF4 confers plasticity in early Th1 cells, poising chromatin specifically at Th17 cell-associated genes. IRF8, an alternative binding partner for BATF, negatively regulates Th17 cell differentiation (Ouyang et al., 2011). Our results identified IRF8 as a member of the Th1 cell transcriptional network whose expression was dependent on RA signaling. Induction of IRF8 would be expected to limit plasticity of Th1 cells by repressing Th17 differentiation, potentially by competing for binding to BATF. In support of a role for IRF8 in regulation of Th1-Th17 axis, patients with mutations in IRF8 have impaired Th1 responses (Hambleton et al., 2011) and single nucleotide polymorphisms (SNPs) in Irf8 are associated with several autoimmune diseases in which IFN-γ⁺ Th17 cells play a pathogenic role (Franke et al., 2010; Graham et al., 2011). It will be of interest to identify transcriptional targets of BATF, IRF4 and IRF8 in Th1 cells.

RA signaling was able to maintain appropriate Th1 cell responses and suppress the development of IL-17⁺ and IFN-γ⁺IL17⁺ cells. Hybrid Th1-Th17 cells are implicated in the pathogenesis of several autoimmune diseases. Their development has been attributed to the plasticity of Th17 cells. Our findings suggest that these cells might alternatively reflect Th1 plasticity and suggest a novel developmental pathway for Th17 cells. Th1 derived ‘Th17’ cells expressed high levels of the receptor for IL-23, a determinant of Th17 pathogenicity (Basu et al., 2013), and were associated with significant gut inflammation and pathology in a model of oral tolerance. Further experiments are required to test the prediction that pathogenic Th17 and IFN-γ⁺IL-17⁺ cells which arise in autoimmunity emerge from Th1 cells when RA is deficient or its signaling perturbed.

A range of inflammatory stimuli can induce RA synthesis and signaling during the course of an immune response. Our results suggest that in a Th1 cell instructing microenvironment the dominant action of A is to repress Th17 cell fate and promote Th1 cell responses. We did not observe enhanced Th17 cell responses during primary Th17 cell differentiation suggesting that the impact of RA on T-cell stability may vary both temporally and among tissues. Previously we have shown in a model of skin allograft rejection that impaired Th1 responses in dnRara mice were accompanied by increased Th2 cell cytokines (Pino-Lagos et al., 2011). We did not identify direct repression of Th2 cell-associated genes by RARα. However, T-bet suppresses GATA3 (Zhu et al., 2012) and in the presence of a Th2 skewing micro-environment, such as the skin, impaired expression of T-bet in the absence of RA signaling renders cells susceptible to Th2 deviation. Thus, the effects of RA on T-cell fate are likely dependent on external and intrinsic factors which shape T-cell polarity. In summary, we show that RA signaling plays a role in regulating stability and functional plasticity of Th1 cells. Regulation of enhancer activity at lineage determining genes by RA-RARα provides mechanistic evidence for reciprocal regulation of Th1 and Th17 cell programs. In the absence of RA signaling, down modulation of T-bet, STAT4 and IFN-γ, and loss of repression of Th17 cell genes, creates a permissive environment for transdifferentiation of Th1 cells to Th17 cells. This study identifies the RA-RARα axis as a potential node for intervention in diseases in which dysregulation of the Th1-Th17 cell axis is observed.

### Example 10

### Embodiments Described Herein

The following embodiments, outline some of the aspects of the technology and approaches described herein:

### Embodiment 1

A method of potentiating anti-tumor immunity in a patient having a tumor comprising

(a) administering an RARα agonist to the patient having a tumor and

(b) providing at least one other therapy to the patient to treat the tumor.

### Embodiment 2

The method of embodiment 1, wherein the at least one other therapy is chosen from:

(a) administering a checkpoint inhibitor to the patient having a tumor;

(b) administering a vaccine to the patient having a tumor; and

(c) treating the patient with T-cell based therapy.

### Embodiment 3

The method of any one of embodiments 1-2, wherein the RARα agonist is chosen from

(a) ATRA

(b) AM580

(c) AM80 (tamibarotene)

(d) BMS753

(e) BD4

(f) AC-93253

(g) AR7

(h) compound of the following formula, or a pharmaceutically acceptable salt thereof:

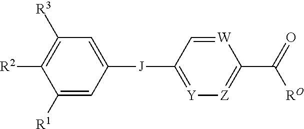

wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R⁴, —O—R^{C}, —O-L-R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

### Embodiment 4

The method of any one of embodiments 1-3, wherein the RAR agonist is a RAMBA.

### Embodiment 5

The method of embodiment 4, wherein the RAMBA is at least one chosen from ketoconazol, liarozol, and tararozol.

### Embodiment 6

The method of any one of embodiments 1-5, wherein the method consolidates and/or maintains Th1 differentiated state in CD4+ and/or CD8+ T-cells.

### Embodiment 7

The method of any one of embodiments 1-6, wherein the RARα agonist is administered without concomitant chemotherapy.

### Embodiment 8

The method of embodiment 7, wherein the patient has had no prior chemotherapy.

### Embodiment 9

The method of embodiment 7, wherein the patient has had no chemotherapy within at least about 2 weeks, 1, 2, or 3 months.

### Embodiment 10

The method of any one of embodiments 7-9, wherein the patient will have no future chemotherapy within at least about 2 weeks, 1, 2, or 3 months.

### Embodiment 11

The method of any one of embodiments 1-10, wherein the at least one other therapy is an immune enhancer.

### Embodiment 12

The method of any one of embodiments 1-11, wherein at least one other therapy promotes Th1 differentiation.

### Embodiment 13

The method of any one of embodiments 1-12, wherein at least one other therapy is used to maintain Th1 immune response.

### Embodiment 14

The method of any one of embodiments 1-13, wherein at least one other therapy is used to reintroduce Th1 immune response.

### Embodiment 15

The method of any one of embodiments 1-14, wherein the Th1 immune response is a Th1 immune response to an antigen expressed by the tumor.

### Embodiment 16

The method of any one of embodiments 1-15, wherein at least one other therapy is a Th1 differentiation therapeutic.

### Embodiment 17

The method of embodiment 16, wherein the Th11 differentiation therapeutic is chosen from IL-12, STAT-4, T-bet, STAT-1, IFN-γ, Runx3, IL-4 repressor, Gata-3 repressor, Notch agonist, and DLL.

### Embodiment 18

The method of any one of embodiments 1-17, wherein at least one other therapy is a checkpoint inhibitor.

### Embodiment 19

The method of embodiment 18, wherein the checkpoint inhibitor is chosen from anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, CD40 agonist, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R checkpoint inhibitor.

### Embodiment 20

The method of embodiment 18, wherein the checkpoint inhibitor causes STAT3 depletion.

### Embodiment 21

The method of embodiment 18, wherein the checkpoint inhibitor is an antibody.

### Embodiment 22

The method of embodiment 19, wherein the antibody checkpoint inhibitor is chosen from an anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, agonistic anti-CD40, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R antibody.

### Embodiment 23

The method of any one of embodiments 18-22, wherein the checkpoint inhibitor helps to induce and/or maintain a therapeutic Th1 response.

### Embodiment 24

The method of any one of embodiments 1-23, wherein at least one other therapy is an antigen, a tumor antigen, and/or a cancer vaccine.

### Embodiment 25

The method of any one of embodiments 1-24, wherein at least one other therapy is a bispecific antibody.

### Embodiment 26

The method of embodiment 25, wherein the bispecific antibody is a bispecific T-cell engaging antibody.

### Embodiment 27

The method of embodiment 26, wherein the bispecific antibody is chosen from anti-CD20 and anti-CD3; anti-CD3 and anti-CD19; anti-EpCAM and anti-CD3; and anti-CEA and anti-CD3.

### Embodiment 28

The method of any one of embodiments 1-27, where at least one other therapy is a T-cell based therapy.

### Embodiment 29

The method of embodiment 28, wherein the T-cell based therapy is ex vivo cell based therapy.

### Embodiment 30

The method of any one of embodiments 1-29, wherein the patient has at least one of melanoma, renal cell cancer, non-small cell lung cancer (including squamous cell cancer and/or adenocarcinoma), bladder cancer, non-Hodgkins lymphoma, Hodgkin's lymphoma, and head and neck cancer.

### Embodiment 31

The method of any one of embodiments 1-29, wherein the patient has adrenocortical carcinoma; AIDS-related cancers (Kaposi sarcoma, lymphoma); anal cancer; appendix cancer; astrocytomas; atypical teratoid/rhabdoid tumor; basal cell carcinoma; bile duct cancer (e.g., extrahepatic bile duct cancer); bladder cancer; bone cancer; Ewing sarcoma family of tumors; osteosarcoma and malignant fibrous histiocytoma; brain stem glioma; brain cancer; central nervous system embryonal tumors; central nervous system germ cell tumors; craniopharyngioma; ependymoma; breast cancer; bronchial tumors; carcinoid tumor; cardiac (heart) tumors; lymphoma, primary; cervical cancer; chordoma; acute myelogenous leukemia (AML); chronic lymphocytic leukemia (CLL); chronic myelogenous leukemia (CML); chronic myeloproliferative neoplasms; colon cancer; colorectal cancer; ductal carcinoma in situ (DCIS); embryonal tumors, endometrial cancer; esophageal cancer; esthesioneuroblastoma; extracranial germ cell tumor; extragonadal germ cell tumor; eye cancer (e.g., intraocular melanoma, retinoblastoma); fallopian tube cancer; gallbladder cancer; gastric (stomach) cancer; gastrointestinal carcinoid tumor; gastrointestinal stromal tumors (GIST); germ cell tumor (e.g., ovarian, testicular); gestational trophoblastic disease; glioma; hairy cell leukemia; head and neck cancer; hepatocellular (liver) cancer; hypopharyngeal cancer; islet-cell tumors, pancreatic cancer (e.g., pancreatic neuroendocrine tumors); kidney cancer (e.g., renal cell, Wilms tumor); Langerhans cell histiocytosis; laryngeal cancer, lip and oral cavity cancer; lung cancer (e.g., non-small cell, small cell); lymphoma (e.g., B-cell, Burkitt, cutaneous T-cell, Sézary syndrome, Hodgkin, non-Hodgkin); primary central nervous system (CNS); male breast cancer; mesothelioma; metastatic squamous neck cancer with occult primary; midline tract carcinoma involving nut gene; mouth cancer; multiple endocrine neoplasia syndromes; multiple myeloma/plasma cell neoplasm; mycosis fungoides; myelodysplastic syndromes; myelodysplastic/myeloproliferative neoplasms; nasal cavity and paranasal sinus cancer; nasopharyngeal cancer, neuroblastoma; oral cancer; oropharyngeal cancer; ovarian cancer (e.g., epithelial tumor, low malignant potential tumor); papillomatosis; paraganglioma; parathyroid cancer; penile cancer; pharyngeal cancer; pheochromocytoma; pituitary tumor; pleuropulmonary blastoma; pregnancy and breast cancer; primary peritoneal cancer; prostate cancer (e.g., castration-resistant prostate cancer); rectal cancer; rhabdomyosarcoma; salivary gland cancer; sarcoma (uterine); skin cancer (e.g., melanoma, Merkel cell carcinoma, nonmelanoma); small intestine cancer; soft tissue sarcoma; squamous cell carcinoma; testicular cancer; throat cancer; thymoma and thymic carcinoma; thyroid cancer; transitional cell cancer of the renal pelvis and ureter; cancer of unknown primary; urethral cancer; uterine cancer, vaginal cancer; vulvar cancer; or Waldenström macroglobulinemia.

### Embodiment 32

The method of embodiment 31, wherein the cancer is chosen from acute myelogenous leukemia, bile duct cancer; bladder cancer; brain cancer; breast cancer; bronchial tumors; cervical cancer; chronic lymphocytic leukemia (CLL); chronic myelogenous leukemia (CML); colorectal cancer; endometrial cancer; esophageal cancer; fallopian tube cancer; gallbladder cancer; gastric (stomach) cancer; head and neck cancer; hepatocellular (liver) cancer; kidney (e.g., renal cell) cancer; lung cancer (non-small cell, small cell); lymphoma (e.g., B-cell); multiple myeloma/plasma cell neoplasm; ovarian cancer (e.g., epithelial tumor); pancreatic cancer; prostate cancer (including castration-resistant prostate cancer); skin cancer (e.g., melanoma, Merkel cell carcinoma); small intestine cancer; squamous cell carcinoma; testicular cancer; cancer of unknown primary; urethral cancer; uterine cancer.

### Embodiment 33

The method of any one of embodiments 1-32, wherein the patient does not have RARα translocated acute myeloid leukemia.

### Embodiment 34

The method of any one of embodiments 1-33, wherein the RARα agonist is not all-trans retinoic acid.

### Embodiment 35

A method of suppressing a Th17 response in a patient comprising administering an RARα agonist and at least one other therapy to the patient.

### Embodiment 36

The method of embodiment 35, wherein the patient has an autoimmune disease and the method treats the autoimmune disease.

### Embodiment 37

The method of any one of embodiments 35-36, wherein the Th17 cells with an IFNg+ and/or IL17+ signature are suppressed.

### Embodiment 38

The method of any one of embodiments 35-37, wherein the RARα agonist is chosen from

(a) ATRA

(b) AM580

(c) AM80 (tamibarotene)

(d) BMS753

(e) BD4

(f) AC-93253

(g) AR7

(h) compound of the following formula, or a pharmaceutically acceptable salt thereof:

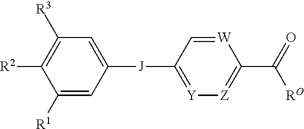

wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R⁴, —O—R^{C}, —O-L-R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

### Embodiment 39

The method of any one of embodiments 35-38, wherein the RARα agonist is coadministered together with a T-cell suppressive agent.

### Embodiment 40

The method of any one of embodiments 35-39, wherein the RARα agonist is coadministered together with abatacept, adalimumab, anakinra, azathioprine, certolizumab, certolizumab pegoltacrolimus, corticosteroids (such as prednisone), dimethyl fumarate, etanercept, fingolimod, glatiramer acetate, golimumab, hydroxychloroquine, infliximab, leflunomide, mercaptopurine, methotrexate, mitoxantrone, natalizumab, rituximab, sulfasalazine, teriflunomide, tocilizumab, tofacitinib, or vedolizumab.

### Embodiment 41

The method of any one of embodiments 35-40, wherein the autoimmune disease is chosen from an autoimmune disease with an IFNg+IL17+ T-cell signature.

### Embodiment 42

The method of any one of embodiments 35-41, wherein the autoimmune disease is chosen from Juvenile Idiopathic Arthritis, Rheumatoid Arthritis, Crohn's disease, and Multiple Sclerosis.

### Embodiment 43

The method of any one of embodiments 35-42, wherein the autoimmune disease is chosen from alopecia areata, autoimmune hemolytic anemia, autoimmune hepatitis, dermatomyositis, type 1 diabetes, juvenile idiopathic arthritis, glomerulonephritis, Graves' disease, Guillain-Barré syndrome, idiopathic thrombocytopenic purpura, myasthenia gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, thyroiditis, uveitis, vitiligo, or granulomatosis with polyangiitis (Wegener's).

### Example 11

### Items Described Herein

While not limiting, certain items are described through the application and in the listing of the following items:

Item 1. A method of potentiating anti-tumor immunity comprising administering an RARα agonist to a patient having a tumor.

Item 2. The method of item 1, wherein the RARα agonist is chosen from
a. ATRA
b. AM580
c. AM80 (tamibarotene)
d. BMS753
e. BD4
f. AC-93253
g. AR7
h. compound of the following formula, or a pharmaceutically acceptable salt thereof:

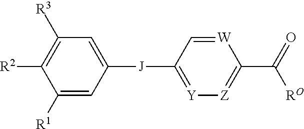

wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R^{A}, —O—R^{C}, —O-L-R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

Item 3. The method of any one of items 1-2, wherein the method consolidates and/or maintains Th1 differentiated state in CD4+ and/or CD8+ T-cells.

Item 4. The method of any one of items 1-3, wherein the RARα agonist is administered without concomitant chemotherapy.

Item 5. The method of item 4, wherein the patient has had no prior chemotherapy.

Item 6. The method of item 4, wherein the patient has had no chemotherapy within at least about 2 weeks, 1, 2, or 3 months.

Item 7. The method of any one of items 4-6, wherein the patient will have no future chemotherapy within at least about 2 weeks, 1, 2, or 3 months.

Item 8. The method of any one of items 1-7, wherein the RARα agonist is administered in combination with at least one other therapy.

Item 9. The method of item 8, wherein the at least one other therapy is an immune enhancer.

Item 10. The method of any one of items 8-9, wherein at least one other therapy promotes Th differentiation.

Item 11. The method of item 10, wherein at least one other therapy is used to maintain Th1 immune response.

Item 12. The method of any one of items 9-11, wherein at least one other therapy is used to reintroduce Th1 immune response.

Item 13. The method of any one of items 11-12, wherein the Th1 immune response is a Th1 immune response to an antigen expressed by the tumor.

Item 14. The method of any one of items 8-13, wherein at least one other therapy is a Th1 differentiation therapeutic.

Item 15. The method of item 14, wherein the Th1 differentiation therapeutic is chosen from IL-12, STAT-4, T-bet, STAT-1, IFN-γ, Runx3, IL-4 repressor, Gata-3 repressor, Notch agonist, and DLL.

Item 16. The method of any one of items 8-15, wherein at least one other therapy is a checkpoint inhibitor.

Item 17. The method of item 16, wherein the checkpoint inhibitor is chosen from anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, anti-CD40, anti-CD40L, anti-GAL9, anti-TIM3, anti-GITR, anti-CD70, anti-CD27, anti-CD137L, anti-CD137, anti-OX40L and anti-OX40 checkpoint inhibitor.

Item 18. The method of item 17, wherein the checkpoint inhibitor is an antibody.

Item 19. The method of any one of items 16-18, wherein the checkpoint inhibitor helps to induce and/or maintain a therapeutic Th1 response.

Item 20. The method of any one of items 8-19, wherein at least one other therapy is an antigen, a tumor antigen, and/or a cancer vaccine.

Item 21. The method of any one of items 1-20, wherein the patient has at least one of melanoma, renal cell cancer, non-small cell lung cancer (including squamous cell cancer and/or adenocarcinoma), bladder cancer, non-Hodgkins lymphoma, Hodgkin's lymphoma, and head and neck cancer.

Item 22. The method of any one of items 1-20, wherein the patient has Adrenocortical Carcinoma; AIDS-Related Cancers (Kaposi Sarcoma, Lymphoma); Anal Cancer; Appendix Cancer; Astrocytomas; Atypical Teratoid/Rhabdoid Tumor; Basal Cell Carcinoma; Bile Duct Cancer; Bladder Cancer; Bone Cancer; Ewing Sarcoma Family of Tumors; Osteosarcoma and Malignant Fibrous Histiocytoma; Brain Stem Glioma; Brain Tumor; Central Nervous System Embryonal Tumors; Central Nervous System Germ Cell Tumors; Craniopharyngioma; Ependymoma; Breast Cancer; Bronchial Tumors; Carcinoid Tumor; Cardiac (Heart) Tumors; Lymphoma, Primary; Cervical Cancer; Chordoma; Chronic Lymphocytic Leukemia (CLL); Chronic Myelogenous Leukemia (CML); Chronic Myeloproliferative Neoplasms; Colon Cancer; Colorectal Cancer; Duct, Bile, Extrahepatic; Ductal Carcinoma In Situ (DCIS); Embryonal Tumors, Endometrial Cancer; Esophageal Cancer; Esthesioneuroblastoma; Extracranial Germ Cell Tumor; Extragonadal Germ Cell Tumor; Eye Cancer (Intraocular Melanoma, Retinoblastoma); Fallopian Tube Cancer; Gallbladder Cancer; Gastric (Stomach) Cancer, Gastrointestinal Carcinoid Tumor; Gastrointestinal Stromal Tumors (GIST); Germ Cell Tumor (Ovarian, Testicular); Gestational Trophoblastic Disease; Glioma; Hairy Cell Leukemia; Head and Neck Cancer; Hepatocellular (Liver) Cancer, Hypopharyngeal Cancer; Islet Cell Tumors, Pancreatic Neuroendocrine Tumors; Kidney (Renal Cell, Wilms Tumor); Langerhans Cell Histiocytosis; Laryngeal Cancer; Lip and Oral Cavity Cancer; Lung Cancer (Non-Small Cell, Small Cell); Lymphoma (Burkitt, Cutaneous T-Cell, Sézary Syndrome, Hodgkin, Non-Hodgkin); Primary Central Nervous System (CNS); Male Breast Cancer; Mesothelioma; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasm; Mycosis Fungoides; Myelodysplastic Syndromes; Myelodysplastic/Myeloproliferative Neoplasms; Nasal Cavity and Paranasal Sinus Cancer; Nasopharyngeal Cancer; Neuroblastoma; Oral Cancer; Oropharyngeal Cancer; Ovarian Cancer (Epithelial Tumor, Low Malignant Potential Tumor); Papillomatosis; Paraganglioma; Parathyroid Cancer; Penile Cancer; Pharyngeal Cancer; Pheochromocytoma; Pituitary Tumor; Pleuropulmonary Blastoma; Pregnancy and Breast Cancer; Primary Peritoneal Cancer; Prostate Cancer; Rectal Cancer; Rhabdomyosarcoma; Salivary Gland Cancer; Sarcoma (Uterine); Skin Cancer (Melanoma, Merkel Cell Carcinoma, Nonmelanoma); Small Intestine Cancer; Soft Tissue Sarcoma; Squamous Cell Carcinoma; Testicular Cancer; Throat Cancer; Thymoma and Thymic Carcinoma; Thyroid Cancer; Transitional Cell Cancer of the Renal Pelvis and Ureter; Unknown Primary; Urethral Cancer; Uterine Cancer, Vaginal Cancer; Vulvar Cancer, or Waldenström Macroglobulinemia.

Item 23. The method of any one of items 1-12, wherein the patient does not have RARα translocated acute myeloid leukemia.

Item 24. The method of any one of items 1-23, wherein the RARα agonist is not all-trans retinoic acid.

Item 25. A method of suppressing a Th17 response in a patient comprising administering an RARα agonist.

Item 26. The method of item 25, wherein the patient has an autoimmune disease.

Item 27. The method of any one of items 25-26, wherein the Th117 cells with an IfNg+ and/or IL17+ signature are suppressed.

Item 28. The method of any one of items 25-27, wherein the RARα agonist is chosen from
a. ATRA
b. AM580
c. AM80 (tamibarotene)
d. BMS753
e. BD4
f. AC-93253
g. AR7
h. compound of the following formula, or a pharmaceutically acceptable salt thereof:

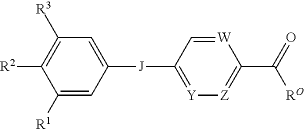

wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R^{A}, —O—R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

Item 29. The method of any one of items 25-28, wherein the RARα agonist is coadministered together with a T-cell suppressive agent.

Item 30. The method of any one of items 25-29, wherein the RARα agonist is coadministered together with abatacept, adalimumab, anakinra, azathioprine, certolizumab, certolizumab pegoltacrolimus, corticosteroids (such as prednisone), dimethyl fumarate, etanercept, fingolimod, glatiramer acetate, golimumab, hydroxychloroquine, infliximab, leflunomide, mercaptopurine, methotrexate, mitoxantrone, natalizumab, rituximab, sulfasalazine, teriflunomide, tocilizumab, tofacitinib, vedolizumab.

Item 31. The method of any one of items 25-30, wherein the autoimmune disease is chosen from an autoimmune disease with an IFNg+IL17+ T-cell signature.

Item 32. The method of any one of items 25-31, wherein the autoimmune disease is chosen from Juvenile Idiopathic Arthritis, Rheumatoid Arthritis, Crohn's disease, and Multiple Sclerosis.

Item 33. The method of any one of items 25-32, wherein the autoimmune disease is chosen from alopecia areata, autoimmune hemolytic anemia, autoimmune hepatitis, dermatomyositis, type 1 diabetes, juvenile idiopathic arthritis, glomerulonephritis, Graves' disease, Guillain-Barré syndrome, idiopathic thrombocytopenic purpura, myasthenia gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, thyroiditis, uveitis, vitiligo, granulomatosis with polyangiitis (Wegener's)

### REFERENCES

Annunziato, F., Cosmi, L., Santarlasci, V., Maggi, L., Liotta, F., Mazzinghi, B., Parente, E., Filì, L, Ferri, S., Frosali, F., et al. (2007). Phenotypic and functional features of human Th17 cells. J Exp Med. 204, 1849-1861.
Aoyama, K., Saha, A., Tolar, J., Riddle, M. J., Veenstra, R. G., Taylor, P. A., Blomhoff, R., Panoskaltsis-Mortari, A., Klebanoff, C. A., Socié, G., et al. (2013). Inhibiting retinoic acid signaling ameliorates graft-versus-host disease by modifying T-cell differentiation and intestinal migration. Blood 122, 2125-2134.
Basu, R. Hatton, R., Weaver, C. (2013) The Th17 family: flexibility follows function. Immunol Rev 252, 89-103.
Chanda, B., Ditadi, A., Iscove, N. N., and Keller, G. (2013). Retinoic Acid Signaling Is Essential for Embryonic Hematopoietic Stem Cell Development. Cell 155, 215-227.
Chen, C., Morris, Q., and Mitchell, J. A. (2012). Enhancer identification in mouse embryonic stem cells using integrative modeling of chromatin and genomic features. BMC Genomics 13, 152-170.
Ciofani, M., Madar, A., Galan, C., Sellars, M., Mace, K., Pauli, F., Agarwal, A., Huang, W., Parkurst, C., Muratet M. et al. (2012). A Validated Regulatory Network for Th17 Cell Specification: Cell 151, 289-303.
Feng, J., Liu, T. and Zhang, Y. (2011) Using MACS to Identify Peaks from ChIP-Seq Data. Current Protocols in Bioinformatics. 34:2.14:2.14.1-2.14.14.
Franke, A., McGovern, D. P. B., Barrett, J. C., Wang, K., Radford-Smith, G. L., Ahmad, T., Lees, C. W., Balschun, T., Lee, J., Roberts, R., et al. (2010). Genome-wide meta-analysis increases to 71 the number of confirmed Crohn's disease susceptibility loci. Nat Genet 42, 1118-1125.
Graham, D. S. C., Morris, D. L., Bhangale, T. R., Criswell, L. A., Syvänen, A.-C., Ronnblom, L., Behrens, T. W., Graham, R. R., and Vyse, T. J. (2011). Association of NCF2, IKZF1, IRF8, IFIH1, and TYK2 with Systemic Lupus Erythematosus. PLoS Genet 7, e1002341.
Hall, J. A., Cannons, J. L., Grainger, J. R., Santos, Dos, L. M., Hand, T. W., Naik, S., Wohlfert, E. A., Chou, D. B., Oldenhove, G., Robinson, M., et al. (2011). Essential Role for Retinoic Acid in the Promotion of CD4+ T-cell Effector Responses via Retinoic Acid Receptor Alpha. Immunity 34, 435-447.
Hambleton, S., Salem, S., Bustamante, J., Bigley, V., Boisson-Dupuis, S., Azevedo, J., Fortin, A., Haniffa, M., Ceron-Gutierrez, L., Bacon, C. M., et al. (2011). IRF8 Mutations and Human Dendritic-Cell Immunodeficiency. N Engl J Med 36.5, 127-138.
Irvin, C., Zafar, 1., Good, J., Rollins, D., Christianson, C., Gorska, M. M., Martin, R. J., and Alam, R. (2014). Increased frequency of dual-positive TH12/TH17 cells in bronchoalveolar lavage fluid characterizes a population of patients with severe asthma. J. Allergy Clin. Immunol. 134, 1175-1186.
Kamei, Y., Xu, L., Heinzel, Y., et al. (1996). A CBP Integrator Complex Mediates Transcriptional Activation and AP-1 Inhibition by Nuclear Receptors, Cell, 85, 403-414.
Kashyap, V., Laursen, K. B., Brenet, F., Viale, A. J., Scandura, J. M., and Gudas, L. J. (2013). RARγ is essential for retinoic acid induced chromatin remodeling and transcriptional activation in embryonic stem cells. J Cell Sci. 126, 999-1008.
Kebir, H., Ifergan, I., Alvarez, J. I., Bernard, M., Poirier, J., Arbour, N., Duquette, P., and Prat, A. (2009). Preferential recruitment of interferon-gamma-expressing TH17 cells in multiple sclerosis. Ann. Neurol. 66, 390-402.
Klebanoff, C. A., Spencer, S. P., Torabi-Parizi, P., Grainger, J. R., Roychoudhuri, R., Ji, Y., Sukumar, M., Muranski, P., Scott, C. D., Hall, J. A., et al. (2013). Retinoic acid controls the homeostasis of pre-cDC-derived splenic and intestinal dendritic cells. J Exp Med. 210, 1961-1976.
Komatsu, N., Okamoto, K., Sawa, S., Nakashima, T., Oh-hora, M., Kodamna, T., Tanaka, S., Bluestone, J. A., and Takayanagi, H. (2014). Pathogenic conversion of Foxp3+ T-cells into TH17 cells in autoimmune arthritis. Nat Med. 20, 62-68.
Larjo, A., Tripathi, S. K., Wagner, U., Luu, Y., and Lönnberg, T. (2013). Global Chromatin State Analysis Reveals Lineage-Specific Enhancers during the Initiation of Human T helper 1 and T helper 2 Cell Polarization: Immunity 38, 1271-1284.
Mucida, D., Park, Y., Kim, G., Turovskaya, O., Scott, I., Kronenberg, M., and Cheroutre, H. (2007). Reciprocal TH17 and regulatory T-cell differentiation mediated by retinoic acid. Science 317, 256.
Mukasa, R., Balasubramani, A., Lee, Y. K., and Whitley, S. K. (2010). Epigenetic Instability of Cytokine and Transcription Factor Gene Loci Underlies Plasticity of the T Helper 17 Cell Lineage. Immunity 28, 616-27.
Nistala, K., Adams, S., Cambrook, H., Ursu, S., Olivito, B., de Jager, W., Evans, J. G., Cimaz, R., Bajaj-Elliott, M., and Wedderburn, L. R. (2010). Th17 plasticity in human autoimmune arthritis is driven by the inflammatory environment. Proc Natl Acad Sci 107, 14751-6.
Ouyang, X., Zhang, R., Yang, J., Li, Q., Qin, L., Zhu, C., Liu, J., Ning, H., Shin, M. S., Gupta, M., et al. (2011). Transcription factor IRF8 directs a silencing programme for TH17 cell differentiation. Nat Commun. 2, 314.
Pino-Lagos, K., Guo, Y., Brown, C., Alexander, M. P., Elgueta, R., Bennett, K. A., De Vries, V., Nowak, E., Blomhoff, R., Sockanathan, S., et al. (2011). A retinoic acid-dependent checkpoint in the development of CD4+ T-cell-mediated immunity. J Exp Med. 208, 1767-1775.
Rada-Iglesias, A., Bajpai, R., Swigut, T., Brugmann, S. A., Flynn, R. A., and Wysocka, J. (2010). A unique chromatin signature uncovers early developmental enhancers in humans. Nature 470, 279-283.
Reis, B. S., Rogoz, A., Costa-Pinto, F. A., Taniuchi, I., and Mucida, D. (2013). Mutual expression of the transcription factors Runx3 and ThPOK regulates intestinal CD4+ T-cell immunity. Nat Immunol. 14, 271-280.
Schraml, B. U., Hildner, K., Ise, W., Lee, W.-L., Smith, W. A. E., Solomon, B., Sahota, G., Sim, J., Mukasa, R., Cemerski, S., et al. (2009). The AP-1 transcription factor Batf controls TH17 differentiation. Nature 460, 405-409.
Schulz, E. G., Mariani, L., Radbruch, A., and Ho □ fer, T. (2009). Sequential polarization and imprinting of type 1 T helper lymphocytes by interferon-gamma and interleukin-12. Immunity 30, 673-683
Spencer, S. P., Wilhelm, C., Yang, Q., Hall, J. A., Bouladoux, N., Boyd, A., Nutman, T. B., Urban, J. F., Wang, J., Ramalingam, T. R., et al. (2014). Adaptation of Innate Lymphoid Cells to a Micronutrient Deficiency Promotes Type 2 Barrier Immunity. Science 343, 432-437.
Takahashi, H., Kanno, T., Nakayamada, S., Hirahara, K., Egrave, G. S., Muljo, S. A., Kuchen, S., Casellas, R., Wei, L., Kanno, Y., et al. (2012). TGF-b and retinoic acid induce the microRNA miR-10a, which targets Bcl-6 and constrains the plasticity of helper T-cells. Nat Immunol. 13, 587-95.
Urvalek, A. M., and Gudas, L. J. (2014). Retinoic Acid and Histone Deacetylases Regulate Epigenetic Changes in Embryonic Stem Cells. J Biol Chem. 289, 19519-19530.
Vahedi, G., Takahashi, H., Nakayamada, S., Sun, H.-W., Sartorelli, V., Kanno, Y., and O'shea, J. J. (2012). STATs shape the active enhancer landscape of T-cell populations. Cell 151, 981-993.
Wadia, J., Stan, R., Dowdy, S. (2004) Transducible TAT-HA fusogenic peptide enhances escape of TAT-fusion proteins after lipid raft macropinocytosis. Nat Med. 10, 310-5.
Wei, L., Vahedi, G., Sun, H.-W., Watford, W. T., Takatori, H., Ramos, H. L., Takahashi, H., Liang, J., Gutierrez-Cruz, G., Zang, C., et al. (2010). Discrete roles of STAT4 and STAT6 transcription factors in tuning epigenetic modifications and transcription during T helper cell differentiation. Immunity 32, 840-851.
Xiao, S., Jin, H., Korn, T., Liu, S. M., Oukka, M., Lim, B., and Kuchroo, V. K. (2008). Retinoic acid increases Foxp3+ regulatory T-cells and inhibits development of Th17 cells by enhancing TGF-beta-driven Smad3 signaling and inhibiting IL-6 and IL-23 receptor expression. J Immunol. 181, 2277-2284.
Yang, Y., Ochando, J. C., Bromberg, J. S., and Ding, Y. (2007). Identification of a distant T-bet enhancer responsive to IL-12/Stat4 and IFN/Stat1 signals. Blood 110, 2494-2500.
Zhang, F., Meng, G., and Strober, W. (2008). Interactions among the transcription factors Runx1, RORgt and Foxp3 regulate the differentiation of interleukin 17-producing T-cells. Nat Immunol. 9, 1297-1306.
Zhang, Y., Liu, T., Meyer, C. A., Eeckhoute, J., Johnson, D. S., Bernstein, B. E., Nusbaum, C., Myers, R. M., Brown, M., Li, W. and Liu, X. S. (2008) Model-based Analysis of ChIP-Seq (MACS). Genome Biology. 9:R 137.
Zhou, L., Ivanov, I. I., Spolski, R., Min, R., Shenderov, K., Egawa, T., Levy, D. E., Leonard, W. J., and Littman, D. R. (2007). IL-6 programs TH-17 cell differentiation by promoting sequential engagement of the IL-21 and IL-23 pathways. Nat. Immunol 13, 587-95.
Zhu, J., Jankovic, D., Oler, A. J., Wei, G., Shanna, S., Hu, G., Guo, L., Yagi, R., Yamane, H., Punkosdy, G., et al. (2012). The Transcription Factor T-bet Is Induced by Multiple Pathways and Prevents an Endogenous Th2 Cell Program during Th1 Cell Responses. Immunity 37, 660-673.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of potentiating anti-tumor immunity in a patient having a tumor comprising
a. administering an RARαt agonist to the patient having a tumor and
b. providing at least one other therapy to the patient to treat the tumor.

2. The method of claim 1, wherein the at least one other therapy is chosen from:
i. administering a checkpoint inhibitor to the patient having a tumor;
ii. administering a vaccine to the patient having a tumor; and
iii. treating the patient with T-cell based therapy.

3. The method of claim 2, wherein the RARα agonist is chosen from
a. ATRA
b. AM580
c. AM80 (tamibarotene)
d. BMS753
e. BD4
f. AC-93253
g. AR7
h. compound of the following formula, or a pharmaceutically acceptable salt thereof: 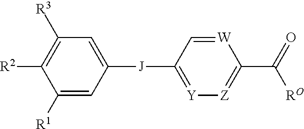
wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R^{A}, —O—R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

4. The method of claim 2, wherein the RAR agonist is a RAMBA.

5. The method of claim 3, wherein the RAMBA is at least one chosen from ketoconazol, liarozol, and tararozol.

6. The method of claim 1, wherein the RARα agonist is administered without concomitant chemotherapy.

7. The method of claim 1, wherein at least one other therapy is a Th1 differentiation therapeutic chosen from IL-12, STAT-4, T-bet, STAT-1, IFN-γ, Runx3, IL-4 repressor, Gata-3 repressor, Notch agonist, and DLL.

8. The method of claim 1, wherein at least one other therapy is a checkpoint inhibitor.

9. The method of claim 8, wherein the checkpoint inhibitor is chosen from anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, CD40 agonist, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R checkpoint inhibitor.

10. The method of claim 8, wherein the checkpoint inhibitor causes STAT3 depletion.

11. The method of claim 8, wherein the checkpoint inhibitor is an antibody chosen from an anti-PD1, anti-PDL1, anti-CD80, anti-CD86, anti-CD28, anti-ICOS, anti-B7RP1, anti-B7H3, anti-B7H4, anti-BTLA, anti-HVEM, anti-LAG-3, anti-CTLA-4, IDO1 inhibitor, agonistic anti-CD40, anti-CD40L, anti-GAL9, anti-TIM3, anti-GITR, anti-CD70, anti-CD27, anti-CD137L, anti-CD137, anti-OX40L, anti-OX40, anti-KIR, anti-B7.1 (also known as anti-CD80), anti-GITR, anti-STAT3, anti CD137 (also known as anti-4-1BB), anti-VISTA, and anti-CSF-1R antibody.

12. The method of claim 1, wherein at least one other therapy is an antigen, a tumor antigen, and/or a cancer vaccine.

13. The method of claim 1, wherein at least one other therapy is a bispecific antibody.

14. The method of claim 13, wherein the bispecific antibody is a bispecific T-cell engaging antibody.

15. The method of claim 14, wherein the bispecific antibody is chosen from anti-CD20 and anti-CD3; anti-CD3 and anti-CD19; anti-EpCAM and anti-CD3; and anti-CEA and anti-CD3.

16. The method of claim 1, where at least one other therapy is a T-cell based therapy.

17. The method of claim 16, wherein the T-cell based therapy is ex vivo cell based therapy.

18. The method of claim 1, wherein the patient has at least one of melanoma, renal cell cancer, non-small cell lung cancer (including squamous cell cancer and/or adenocarcinoma), bladder cancer, non-Hodgkins lymphoma, Hodgkin's lymphoma, and head and neck cancer.

19. The method of claim 1, wherein the patient has adrenocortical carcinoma; AIDS-related cancers (Kaposi sarcoma, lymphoma); anal cancer; appendix cancer; astrocytomas; atypical teratoid/rhabdoid tumor; basal cell carcinoma; bile duct cancer (e.g., extrahepatic bile duct cancer); bladder cancer; bone cancer; Ewing sarcoma family of tumors; osteosarcoma and malignant fibrous histiocytoma; brain stem glioma; brain cancer; central nervous system embryonal tumors; central nervous system germ cell tumors; craniopharyngioma; ependymoma; breast cancer; bronchial tumors; carcinoid tumor; cardiac (heart) tumors; lymphoma, primary; cervical cancer; chordoma; acute myelogenous leukemia (AML); chronic lymphocytic leukemia (CLL); chronic myelogenous leukemia (CML); chronic myeloproliferative neoplasms; colon cancer; colorectal cancer; ductal carcinoma in situ (DCIS); embryonal tumors, endometrial cancer; esophageal cancer; esthesioneuroblastoma; extracranial germ cell tumor; extragonadal germ cell tumor; eye cancer (e.g., intraocular melanoma, retinoblastoma); fallopian tube cancer; gallbladder cancer; gastric (stomach) cancer; gastrointestinal carcinoid tumor; gastrointestinal stromal tumors (GIST); germ cell tumor (e.g., ovarian, testicular); gestational trophoblastic disease; glioma; hairy cell leukemia; head and neck cancer; hepatocellular (liver) cancer; hypopharyngeal cancer; islet-cell tumors, pancreatic cancer (e.g., pancreatic neuroendocrine tumors); kidney cancer (e.g., renal cell, Wilms tumor); Langerhans cell histiocytosis; laryngeal cancer; lip and oral cavity cancer; lung cancer (e.g., non-small cell, small cell); lymphoma (e.g., B-cell, Burkitt, cutaneous T-cell, Sézary syndrome, Hodgkin, non-Hodgkin); primary central nervous system (CNS); male breast cancer; mesothelioma; metastatic squamous neck cancer with occult primary; midline tract carcinoma involving nut gene; mouth cancer; multiple endocrine neoplasia syndromes; multiple myeloma/plasma cell neoplasm; mycosis fungoides; myelodysplastic syndromes; myelodysplastic/myeloproliferative neoplasms; nasal cavity and paranasal sinus cancer; nasopharyngeal cancer; neuroblastoma; oral cancer; oropharyngeal cancer; ovarian cancer (e.g., epithelial tumor, low malignant potential tumor); papillomatosis; paraganglioma; parathyroid cancer; penile cancer; pharyngeal cancer; pheochromocytoma; pituitary tumor; pleuropulmonary blastoma; pregnancy and breast cancer, primary peritoneal cancer; prostate cancer (e.g., castration-resistant prostate cancer); rectal cancer; rhabdomyosarcomna; salivary gland cancer, sarcoma (uterine); skin cancer (e.g., melanoma, Merkel cell carcinoma, nonmelanoma); small intestine cancer; soft tissue sarcoma; squamous cell carcinoma; testicular cancer; throat cancer; thymoma and thymic carcinoma; thyroid cancer; transitional cell cancer of the renal pelvis and ureter; cancer of unknown primary; urethral cancer; uterine cancer, vaginal cancer; vulvar cancer; or Waldenström macroglobulinemia.

20. The method of claim 19, wherein the cancer is chosen from acute myelogenous leukemia, bile duct cancer; bladder cancer; brain cancer; breast cancer; bronchial tumors; cervical cancer; chronic lymphocytic leukemia (CLL); chronic myelogenous leukemia (CML); colorectal cancer; endometrial cancer; esophageal cancer; fallopian tube cancer; gallbladder cancer; gastric (stomach) cancer; head and neck cancer; hepatocellular (liver) cancer; kidney (e.g., renal cell) cancer; lung cancer (non-small cell, small cell); lymphoma (e.g., B-cell); multiple myeloma/plasma cell neoplasm; ovarian cancer (e.g., epithelial tumor); pancreatic cancer; prostate cancer (including castration-resistant prostate cancer); skin cancer (e.g., melanoma, Merkel cell carcinoma); small intestine cancer; squamous cell carcinoma; testicular cancer; cancer of unknown primary; urethral cancer; uterine cancer.

21. The method of claim 1, wherein the patient does not have RARα translocated acute myeloid leukemia.

22. The method of claim 1, wherein the RARα agonist is not all-trans retinoic acid.

23. A method of suppressing a Th17 response in a patient comprising administering an RARα agonist and at least one other therapy to the patient.

24. The method of claim 23, wherein the patient has an autoimmune disease and the method treats the autoimmune disease.

25. The method of claim 23, wherein the Th17 cells with an IFNg+ and/or IL17+ signature are suppressed.

26. The method of claim 23, wherein the RARα agonist is chosen from
a. ATRA
b. AM580
c. AM80 (tamibarotene)
d. BMS753
e. BD4
f. AC-93253
g. AR7
h. compound of the following formula, or a pharmaceutically acceptable salt thereof: 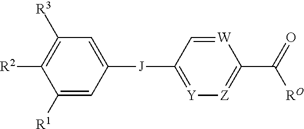
wherein: —R¹ is independently —X, —R^{X}, —O—R^{X}, —O—R^{A}, —O—R^{C}, —O-L-R^{C}, —O—R^{AR}, or —O-L-R^{AR}; —R² is independently —X, —R^{X}, —O—R^{X}, —O—R^{A}, —O—R^{C}, —O-L-R^{C}, —O—R^{AR}, or —O-L-R^{AR}; —R³ is independently —X, —R^{X}, —O—R^{X}, —O—R^{A}, —O—R^{C}, —O-L-R^{C}, —O—R^{AR}, or —O-L-R^{AR}; with the proviso that —R¹, —R², and —R³ are not all —O—R^{A}; wherein: each —X is independently —F, —Cl, —Br, or —I; each —R^{A} is saturated aliphatic C₁₋₆alkyl; each —R^{X} is saturated aliphatic C₁₋₆haloalkyl; each —R^{C} is saturated C₃₋₇cycloalkyl; each —R^{AR} is phenyl or C₅₋₆heteroaryl; each -L- is saturated aliphatic C₁₋₃alkylene; and wherein: -J- is —C(═O)—NR^{N}—; —R^{N} is independently —H or —H or —R^{NN}; —R^{NN} is saturated aliphatic C₁₋₄alkyl; ═Y— is ═CR^{Y}— and —Z═ is —CR^{Z}═; —R^{Y} is —H; —R^{Z} is independently —H or —R^{ZZ}; —R^{ZZ} is independently —F, —Cl, —Br, —I, —OH, saturated aliphatic C₁₋₄alkoxy, saturated aliphatic C₁₋₄alkyl, or saturated aliphatic C₁₋₄haloalkyl; ═W— is ═CR^{W}—; —R^{W} is —H; —R^{O} is independently —OH, —OR^{E}, —NH₂, —NHR^{T1}, —NR^{T1}R^{T1} or —NR^{T2}R^{T3}; —R^{E} is saturated aliphatic C₁₋₆alkyl; each —R^{T1} is saturated aliphatic C₁₋₆alkyl; —NR^{T2}R^{T3} is independently azetidino, pyrrolidino, piperidino, piperizino, N—(C₁₋₃alkyl) piperizino, or morpholino; with the proviso that the compound is not a compound selected from the following compounds, and salts, hydrates, and solvates thereof: 4-(3,5-dichloro-4-ethoxy-benzoylamino)-benzoic acid (PP-02); and 4-(3,5-dichloro-4-methoxy-benzoylamino)-benzoic acid (PP-03).

27. The method of claim 23, wherein the RARα agonist is coadministered together with a T-cell suppressive agent.

28. The method of claim 23, wherein the RARα agonist is coadministered together with abatacept, adalimumab, anakinra, azathioprine, certolizumab, certolizumab pegoltacrolimnus, corticosteroids (such as prednisone), dimethyl fumarate, etanercept, fingolimod, glatiramer acetate, golimumab, hydroxychloroquine, infliximab, leflunomide, mercaptopurine, methotrexate, mitoxantrone, natalizumab, rituximab, sulfasalazine, teriflunomide, tocilizumab, tofacitinib, or vedolizumab.

29. The method of claim 23, wherein the autoimmune disease is chosen from an autoimmune disease with an IFNg+IL17+ T-cell signature.

30. The method of claim 23, wherein the autoimmune disease is chosen from juvenile idiopathic arthritis, rheumatoid arthritis, Crohn's disease, multiple sclerosis, alopecia areata, autoimmune hemolytic anemia, autoimmune hepatitis, dermatomyositis, type 1 diabetes, juvenile idiopathic arthritis, glomerulonephritis, Graves' disease, Guillain-Barré syndrome, idiopathic thrombocytopenic purpura, myasthenia gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, thyroiditis, uveitis, vitiligo, or granulomatosis with polyangiitis (Wegener's).


SEQUENCE LISTING (24 sequences)

SEQ ID NO: 1 - DNA, 20 bp, Artificial Sequence
  Stat4_+105k F primer
tcctcctccctttgttgttc

SEQ ID NO: 2 - DNA, 20 bp, Artificial Sequence
  Stat4_+105k F primer
gggccttaatcaaccatttc

SEQ ID NO: 3 - DNA, 20 bp, Artificial Sequence
  Stat4 Promoter F primer
agagggcatacaccgagaac

SEQ ID NO: 4 - DNA, 20 bp, Artificial Sequence
  Stat4 Promoter R primer
tctagggagccagcatcaac

SEQ ID NO: 5 - DNA, 19 bp, Artificial Sequence
  Tbx21 Promoter F primer
tcgcttttggtgaggactg

SEQ ID NO: 6 - DNA, 20 bp, Artificial Sequence
  Tbx21 Promoter R primer
ggtggcaggttgactctttc

SEQ ID NO: 7 - DNA, 20 bp, Artificial Sequence
  Tbx21 -12k F primer
gcggaagagggaactaacac

SEQ ID NO: 8 - DNA, 20 bp, Artificial Sequence
  Tbx21 -12k R primer
ggacccggaacctatgtatg

SEQ ID NO: 9 - DNA, 20 bp, Artificial Sequence
  Irf8 Promoter F primer
cagaagctagggctggtgtc

SEQ ID NO: 10 - DNA, 22 bp, Artificial Sequence
  Irf8 Promoter R primer
cacagaacagatcccaaatgtc

SEQ ID NO: 11 - DNA, 20 bp, Artificial Sequence
  Irf8 -11k F primer
ccttaaccccggaactgtag

SEQ ID NO: 12 - DNA, 20 bp, Artificial Sequence
  Irf8 -11k R primer
tgctgtgcttgcctctactc

SEQ ID NO: 13 - DNA, 20 bp, Artificial Sequence
  Il6ra Promoter F primer
tccgcttgagttttgctttc

SEQ ID NO: 14 - DNA, 24 bp, Artificial Sequence
  Il6ra Promoter R primer
cactgacctgccttctactttaac

SEQ ID NO: 15 - DNA, 23 bp, Artificial Sequence
  Il6ra +32k F primer
caaagctaaaaccaggaaatgac

SEQ ID NO: 16 - DNA, 21 bp, Artificial Sequence
  Il6ra +32k R primer
aaaaggttccatgtgatgttg

SEQ ID NO: 17 - DNA, 20 bp, Artificial Sequence
  Rorc Promoter (Ror-gamma-t isoform) F primer
aggaatttgggtgtggtgag

SEQ ID NO: 18 - DNA, 20 bp, Artificial Sequence
  Rorc Promoter (Ror-gamma-t isoform) R primer
ctgtcttgggtggtgtcttg

SEQ ID NO: 19 - DNA, 20 bp, Artificial Sequence
  Runx1 Promoter 1 F primer
tggaagaggaagaagctgtg

SEQ ID NO: 20 - DNA, 20 bp, Artificial Sequence
  Runx1 Promoter 1 R primer
caagagaagccaccccaaac

SEQ ID NO: 21 - DNA, 21 bp, Artificial Sequence
  Runx1 Promoter 2 F primer
tgctgggcttacacttctgac

SEQ ID NO: 22 - DNA, 22 bp, Artificial Sequence
  Runx1 Promoter 2 R primer
tggacctcataaacaaccacag

SEQ ID NO: 23 - DNA, 21 bp, Artificial Sequence
  IFNg +28k F primer
ctttgagccactgatgggtag

SEQ ID NO: 24 - DNA, 21 bp, Artificial Sequence
  IFNg +28k R primer
gcctctccacgtctcttcttc